(19) United States
(12) Patent Application Publication
(10) Pub. No.: US 2025/0332267 A1
(43) Pub. Date: Oct. 30, 2025

(54) DRUG DELIVERY

(71) Applicant: Oxford University Innovation Limited, Oxford (GB)

(72) Inventors: Daniel ANTHONY, Oxford (Oxfordshire) (GB); Ben DAVIS, Oxford (Oxfordshire) (GB); Kim WALS, Oxford (Oxfordshire) (GB); Naveed AKBAR, Oxford (Oxfordshire) (GB); Megan SEALEY, Oxford (Oxfordshire) (GB)

(21) Appl. No.: 18/774,144

(22) Filed: Jul. 16, 2024

Related U.S. Application Data
(63) Continuation of application No. 17/051,507, filed on Oct. 29, 2020, now abandoned.

(30) Foreign Application Priority Data
Apr. 30, 2018 (GB) 1807040.9

(51) Int. Cl. A61K 47/54; A61K 45/06; A61K 47/69; B82Y 5/00

(52) U.S. Cl. CPC A61K 47/549; A61K 45/06; A61K 47/6911; B82Y 5/00

(57) ABSTRACT

A drug delivery vehicle comprising a vesicle conjugated to one or more targeting groups, wherein the targeting groups comprise an oligosaccharide which is Lewis A or Lewis B or a mimetic thereof, or a pharmaceutically acceptable salt or PEGylated form of the oligosaccharide:

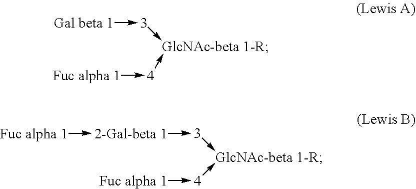

wherein R represents the point of attachment to the vesicle.

### FIELD OF THE INVENTION

This invention relates to drug delivery vehicles suitable for delivery of drugs to the brain. The invention also relates to such drug delivery vehicles containing drugs suitable for treating diseases and disorders of the brain, and to the use of the drug delivery vehicles in treating brain disease or disorder.

### BACKGROUND TO THE INVENTION

A major challenge of neuropathology is how to selectively deliver drugs to the brain in the presence of the blood-brain barrier (BBB), which exists between the central nervous system and peripheral circulation. Comprised of microvascular endothelial cells, continuous tight junctions, astrocytic end feet, and pericytes, the BBB is normally tightly regulated. Even low molecular weight species which are highly soluble and may initially cross the BBB are often excluded or pumped out of the brain by transporters. High molecular weight species generally will not pass across the BBB, so delivery of biologic agents such as antibodies, to treat brain diseases and disorders, has been difficult to achieve.

Various strategies have been used in the past to try and increase movement of such high molecular weight species across the BBB. For instance, biologic drugs have been tagged with, for example, transferrin or antibodies that target the transferrin receptor. Transport into the brain can then be achieved via transport on the endogenous blood brain barrier transferrin receptor. Similarly, others have found that tagging with glutathione can increase transport across BBB.

However, these strategies lead to transport of active agent indiscriminately into the brain. This can lead to an array of unwanted effects as active biologic agents are delivered to un-diseased areas of the brain. There is therefore a need for more targeted techniques for delivering active agent to the brain.

One particular area of research into central nervous system (CNS) related disorders is the accumulation of tau into pathological aggregates, which is a hallmark found in over 20 different diseases, collectively known as tauopathies, such as Alzheimer's Disease (AD), Progressive supranuclear palsy, and Frontotemporal dementia. In AD, pathological tau aggregates primarily appear in the entorhinal cortex and subsequently spread in a hierarchical pattern to the hippocampus and neocortex. This process of tau propagation correlates strongly with severity of symptoms in patients. Recent findings demonstrated that toxic tau aggregates can transmit across synaptically connected neurons and that there are specific tau ‘strains’ that have increased ability to propagate tau pathology in vivo. A potential mechanism for the transmission of tau across synapses is through exosomes. Stereotaxic injections of tau-containing exosomes (TcEs), into the brains of naïve mice has been shown to induce tau pathology. Interestingly, this exosome-mediated tau propagation appears to be dependent on microglial activity. When microglia are depleted from the brain, tau propagation is abolished, indicating a role for inflammation in tau pathology. Due to these recent advancements into research in tau propagation, there has been a push to find therapeutic interventions that target tau pathology in the brain. However, the presence of the blood-brain barrier remains an obstacle for the delivery of such large or polar molecules.

Various strategies to target tau with therapeutic agents are currently being investigated, such as the degradation of tau by the proteasome. Recently, Otub1 has been identified as a tau-deubiquitinating enzyme (DUB) that impairs the degradation of tau. Otub1 was also shown to induce tau-seeded aggregation in vitro and in vivo. Inhibitors of DUBs, such as VLX1570, are currently under investigation for the treatment of myeloma cancer, and are demonstrating promising results. Therefore, it is highly possible that these types of drugs have the potential to treat other diseases, such as AD. However, selectively delivering these drugs to tau in the brain remains a challenge.

There is therefore a need for improved drug delivery mechanisms to introduce drugs to the brain, in particular to provide drugs in a more targeted fashion than has previously been available.

### SUMMARY OF THE INVENTION

The present inventors have surprisingly found that vesicles containing active agents for treatment of brain disease can be delivered selectively to active sites in the brain by conjugation to specific targeting oligosaccharides. These targeting oligosaccharides are Lewis A or Lewis B saccharides, or mimetics thereof, pharmaceutically acceptable salts of Lewis A, Lewis B or their mimetics, or PEG-ylated forms of Lewis A, Lewis B or their mimetics or salts.

Lewis A and Lewis B have previously been used in imaging of the endothelium in the brain. However it has now been surprisingly found that Lewis A and Lewis B bind to cell adhesion molecules, for example E-selectin and P-selectin, in particular E-selectin, a cell adhesion molecule expressed on endothelial cells activated by cytokines. E-selectin is known to play an important part in inflammation and has been used as a marker for inflammation in acute inflammation in the brain. Cell adhesion molecules such as E-selectin are transported across the blood brain barrier and into microglial cells. Accordingly, binding to E-selectin or other cell adhesion molecules can lead to transport across the blood brain barrier and targeting to focal sites of otherwise inaccessible brain pathology. This finding that Lewis A and Lewis B and their mimetics are transported into brain cells themselves, rather than merely to endothelial cells, was not predictable from earlier results and has led to the present invention.

In accordance with this finding, the present inventors have found that vesicles containing drug payload can be conjugated to Lewis A or Lewis B and selectively targeted to cell adhesions molecules, in particular E-selectin. Administration of these targeted drug delivery vehicles will therefore enable drug to cross the BBB and accumulate at sites of interest, not only in activated endothelial cells themselves, but also brain parenchymal cells, in particular in microglial cells. This leads to particular value in delivering drugs, including high molecular weight biologics, selectively to the brain in the treatment of various brain diseases and disorders.

Accordingly, the present invention provides a drug delivery vehicle comprising a vesicle conjugated to one or more targeting groups, wherein the targeting groups comprise an oligosaccharide which is Lewis A or Lewis B or a mimetic thereof, or a pharmaceutically acceptable salt or PEGylated form of the oligosaccharide:

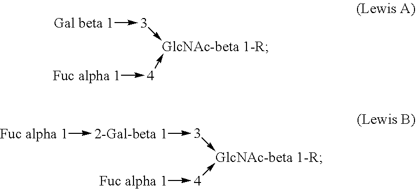

wherein R represents the point of attachment to the vesicle.

In an alternative embodiment, the drug delivery vehicle is a drug delivery vehicle comprising a vesicle conjugated to one or more targeting groups, wherein the targeting groups comprise an oligosaccharide of formula (I), or a pharmaceutically acceptable salt or PEGylated form of the oligosaccharide:

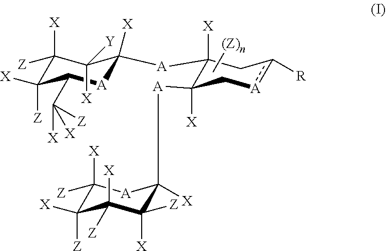

wherein each Z is the same or different and is selected from OH, hydrogen, halogen, C₁₋₆alkoxy, —NR′R″, —NR′COR″, —N(COR′)(COR″), —SR′, —COR′, —COOR′, —OC(O)R′, —OC(O)OR′, —OC(O)NR′R″, —OC(O)SR′, —OP(O)(OR′)(OR″), —OSO₃H, or C₁₋₁₂ alkyl, C₂₋₁₂ alkenyl or C₂₋₁₂ alkynyl, which is optionally substituted with one or more substituents selected from halogen, NH₂, N₃, CN, COOH, COO(C₁₋₄ alkyl), OH and C₁₋₄ alkoxy; each X is the same or different and is selected from OH, hydrogen, halogen, C₁₋₆alkoxy, —NR′R″, —NR′COR″, —N(COR′)(COR″), —SR′, —COR′, —COOR′, —OC(O)R′, —OC(O)OR′, —OC(O)NR′R″, —OC(O)SR′, —OP(O)(OR′)(OR″), —OSO₃H, or C₁₋₁₂ alkyl, C₂₋₁₂ alkenyl or C₂₋₁₂ alkynyl, which is optionally substituted with one or more substituents selected from halogen, NH₂, N₃, CN, COOH, COO(C₁₋₄ alkyl), OH and C₁₋₄ alkoxy; each A is the same or different and is selected from CR′R″, O, S and NR′;
n is 0, 1, 2, 3 or 4;
Y is either a group Z as defined above or a saccharide unit of formula (II):

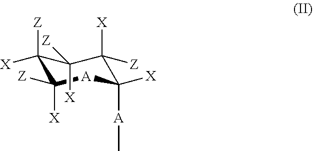

wherein X, Z and A are as defined above;
R represents the point of attachment to the vesicle; and
R′ and R″ are identical or different and are selected from hydrogen and C₁₋₁₂ alkyl groups which are optionally substituted with one or more substituents selected from halogen, NH₂, N₃, CN, COOH, COO(C₁₋₄ alkyl), OH and C₁₋₄ alkoxy.

Typically, each Z is the same or different and is selected from OH, hydrogen, halogen, C₁₋₆alkoxy, —NR′R″, —NR′COR″, —N(COR′)(COR″), —SR′, —COR′, —COOR′, —OC(O)R′, —OC(O)OR′, —OC(O)NR′R″, —OC(O)SR′, —OP(O)(OR′)(OR″), or C₁₋₁₂ alkyl, C₂₋₁₂ alkenyl or C₂₋₁₂ alkynyl, which is optionally substituted with one or more substituents selected from halogen, NH₂, N₃, CN, COOH, COO(C₁₋₄ alkyl), OH and C₁₋₄ alkoxy; and
each X is the same or different and is selected from OH, hydrogen, halogen,
C₁₋₆alkoxy, —NR′R″, —NR′COR″, —N(COR′)(COR″), —SR′, —COR′, —COOR′, —OC(O)R′, —OC(O)OR′, —OC(O)NR′R″, —OC(O)SR′, —OP(O)(OR′)(OR″), or C₁₋₁₂ alkyl, C₂₋₁₂ alkenyl or C₂₋₁₂ alkynyl, which is optionally substituted with one or more substituents selected from halogen, NH₂, N₃, CN, COOH, COO(C₁₋₄ alkyl), OH and C₁₋₄ alkoxy.

Also provided is a pharmaceutical composition comprising a drug delivery vehicle of the invention and a pharmaceutically acceptable carrier or diluent.

The drug delivery vehicles may contain active therapeutic agents which are useful in treating brain disease, in particular diseases where selective targeting of active endothelial cells or microglial cells in the proximity of the vasculature is desired. For instance, the drug delivery vehicles may contain drugs for treating Alzheimer's disease, multiple sclerosis and brain tumour including metastasis. Therapeutic agents useful in the treatment of Alzheimer's disease which may be included in the drug delivery vehicles of the invention include deubiquitinating enzyme inhibitors.

Accordingly, the invention also provides drug delivery vehicles wherein the vesicle comprises a therapeutic agent for use in treating a disease or disorder of the brain, for example a therapeutic agent for use in treating Alzheimer's disease, multiple sclerosis, brain tumour or metastasis, for example a deubiquitinating enzyme inhibitor.

The invention also provides drug delivery vehicles and pharmaceutical compositions wherein the vesicle contains an active therapeutic agent for use in a method of treatment or prevention of a disease or disorder of the brain. Also provided are methods for treatment or prevention of a disease or disorder of the brain in a subject, the method comprising administering to the subject an effective amount of a drug delivery vehicle as described herein, wherein the vesicle contains an active therapeutic agent, or a pharmaceutical composition containing such a drug delivery vehicle. Also provided is the use of a drug delivery vehicle as described herein, wherein the vesicle contains an active therapeutic agent, or a pharmaceutical composition containing such a drug delivery vehicle, in the manufacture of a medicament for the treatment or prevention of a brain disease or disorder.

### BRIEF DESCRIPTION OF THE FIGURES

FIG. 1 shows schematically the construction of the drug delivery vehicle of the invention.

FIG. 2A provides quantification of Lewis A-MPIO (magnetic particles of iron oxide) binding events in Selectin-blocked and isotype control mice; FIG. 2B shows analysis of only the basal ganglia (BG) for Lewis A-MPIO binding; FIGS. 2C and D provide equivalent results for Lewis B-MPIO. Analysis carried out using 1 way ANOVA using a Bonferroni's Multiple Comparison Test (*P≤0.05, **P≤0.01, ***P≤0.001, Numbers: n=4 for all, except E-Selectin Block Lewis B and Isotype Control II Lewis B which are n=3.

FIG. 3A provides quantification of Lewis A-MPIO binding events in control and neutrophil depleted mice; FIG. 3B provides analysis of only the basal ganglia (BG) for Lewis A-MIPO binding; FIGS. 3C and D provide equivalent results for Lewis B-MP10. Analysis carried out using two-tailed unpaired t test. (*P≤0.05, **P≤0.01, ***P≤0.001). Numbers: n=4 for all.

FIG. 4 shows accumulation of targeted liposomes of the invention following administration to mice by IV injection.

FIG. 5 shows the fold change in VCAM-1 mRNA following incubation of activated mouse brain endothelium with a Lewis A labelled liposome composition transfected with siRNA. Comparison of liposomes transfected with a final concentration of 25 nM siRNA, or Lewis A labelled liposomes transfected with an equivalent concentration of siRNA.

### DETAILED DESCRIPTION OF THE INVENTION

As used herein, an alkyl group or moiety is a linear or branched alkyl group or moiety containing from 1 to 12, preferably from 1 to 8, for example from 1 to 6, carbon atoms such as a C₁₋₄ alkyl group or moiety. Examples of C₁₋₄ alkyl groups and moieties include methyl, ethyl, n-propyl, i-propyl, n-butyl, i-butyl and t-butyl. For the avoidance of doubt, where two alkyl moieties are present in a group, the alkyl moieties may be the same or different.

As used herein, an alkenyl group or moiety is a linear or branched alkenyl group or moiety containing from 2 to 12, preferably from 2 to 8, for example from 2 to 6, carbon atoms such as a C₂₋₄ alkenyl group or moiety. Examples of C₂₋₄ alkenyl groups and moieties include ethenyl, propenyl, and butenyl. For the avoidance of doubt, where two alkenyl moieties are present in a group, the alkenyl moieties may be the same or different.

As used herein, an alkynyl group or moiety is a linear or branched alkynyl group or moiety containing from 2 to 12, preferably from 2 to 8, for example from 2 to 6, carbon atoms such as a C₂₋₄ alkynyl group or moiety. Examples of C₂₋₄ alkynyl groups and moieties include ethynyl, propynyl and butynyl. For the avoidance of doubt, where two alkynyl moieties are present in a group, the alkynyl moieties may be the same or different.

An alkyl, alkenyl or alkynyl group as used herein may be unsubstituted or substituted. For example it may be substituted with up to four, for example one, two or three, substituents selected from halogen, NH₂, N₃, CN, COOH, COO(C₁₋₄ alkyl), OH and C₁₋₄ alkoxy. Preferred substituents are halogen, NH₂, OH and C₁₋₂ alkoxy. The substituents are themselves unsubstituted. Typically, an alkyl, alkenyl or alkynyl group as used herein is unsubstituted or substituted with one substituent. Preferably it is unsubstituted.

As used herein the term amino represents a group of formula —NH₂. The term C₁₋₂ alkylamino represents a group of formula —NHR′ wherein R′ is methyl or ethyl. The term di(C₁₋₂ alkyl)amino represents a group of formula —NR′R″ wherein R′ and R″ are the same or different and represent methyl or ethyl. As used herein a C₁₋₂ acetylamino group is a C₁₋₂ acetyl group attached to an amino group as defined above. Similarly, a di(C₁₋₂)acetylamino group is an amino group bearing two C₁₋₂ acetyl groups.

As used herein, an alkoxy group is typically a said alkyl group attached to an oxygen atom. Similarly, an alkylthio group is typically a said alkyl group attached to a thio group.

A used herein halogen is typically fluorine, chlorine, bromine or iodine, preferably fluorine or chlorine, most preferably fluorine.

### Targeting Groups

The drug delivery vehicles of the invention comprise a vesicle, which may contain an active therapeutic agent, which is conjugated to one or more targeting groups. One or more of the targeting groups comprises an oligosaccharide which is a Lewis A or Lewis B oligosaccharide or a mimetic thereof, as described herein. Where two or more targeting groups comprising oligosaccharides are present, oligosaccharide moieties may be the same or different. Preferably, the oligosaccharide is Lewis A (Le^{a}) or Lewis B (Le^{b}), most preferably Le^{a}. Where the oligosaccharide moiety is Le^{a} or Le^{b}, it can be represented as follows:

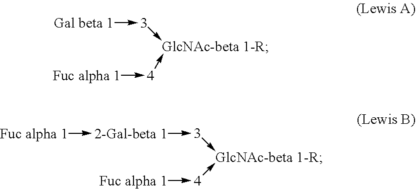

wherein R represents the point of attachment to the vesicle.

Mimetics of Le^{a} and Le^{b} can also be used. Typically, the mimetics will contain one or more modifications compared with the basic oligosaccharide structures. For example, the mimetic may contain one, two, three or four modifications compared with the basic oligosaccharide structure. Typically, each saccharide unit within the Le^{a} or Le^{b} unit contains none, one or two, preferably none or one modification.

The mimetic of Lewis A or Lewis B may, for example, be Lewis A or Lewis B having one or more of the following modifications (i) to (iv):
(i) one or more OH and/or NAc groups are independently replaced with hydrogen, halogen, C₁₋₆ alkoxy, —NR′R″, —NR′COR″, —N(COR′)(COR″), —SR′, —COR′, —COOR′, —OC(O)R′, —OC(O)OR′, —OC(O)NR′R″, —OC(O)SR′, —OP(O)(OR′)(OR″), —OSO₃H, or C₁₋₂ alkyl, C₂₋₁₂ alkenyl or C₂₋₁₂ alkynyl which is optionally substituted with one or more substituents selected from halogen, NH₂, N₃, CN, COOH, COO(C₁₋₄ alkyl), OH and C₁₋₄ alkoxy, or in the case of NAc may be replaced with OH;
(ii) one or more hydrogen atoms are independently replaced with OH, halogen or a group selected from OR′, —NR′R″, —SR′, —COR′, —COOR′, —OC(O)R′, —OC(O)OR′, —OC(O)NR′R″, —OC(O)SR′, —OP(O)(OR′)(OR″), —OSO₃H, or C₁₋₁₂ alkyl, C₂₋₁₂ alkenyl or C₂₋₁₂ alkynyl which is optionally substituted with one or more substituents selected from halogen, NH₂, N₃, CN, COOH, COO(C₁₋₄ alkyl), OH and C₁₋₄ alkoxy;
(iii) one or more —O— moieties are independently replaced with —CR′R″—, —S—, —NR′— or —NR′CO—;
(iv) in the Glc saccharide unit, the unit —C₁(R)—O— is replaced with —C₁(R)═N— or —C₁(R)═CR′—;
wherein each R′ and R″ is identical or different and is selected from hydrogen and C₁₋₁₂ alkyl groups which are optionally substituted with one or more substituents selected from halogen, NH₂, N₃, CN, COOH, COO(C₁₋₄ alkyl), OH and C₁₋₄ alkoxy.

Typically, modification (i) is as follows:
(i) one or more OH and/or NAc groups are independently replaced with hydrogen, halogen, C₁₋₆alkoxy, —NR′R″, —NR′COR″, —N(COR′)(COR″), —SR′, —COR′, —COOR′, —OC(O)R′, —OC(O)OR′, —OC(O)NR′R″, —OC(O)SR′, —OP(O)(OR′)(OR″), or C₁₋₁₂ alkyl, C₂₋₁₂ alkenyl or C₂₋₁₂ alkynyl which is optionally substituted with one or more substituents selected from halogen, NH₂, N₃, CN, COOH, COO(C₁₋₄ alkyl), OH and C₁₋₄ alkoxy, or in the case of NAc may be replaced with OH; and
   modification (ii) is as follows:
(ii) one or more hydrogen atoms are independently replaced with OH, halogen or a group selected from OR′, —NR′R″, —SR′, —COR′, —COOR′, —OC(O)R′, —OC(O)OR′, —OC(O)NR′R″, —OC(O)SR′, —OP(O)(OR′)(OR″), or C₁₋₁₂ alkyl, C₂₋₁₂ alkenyl or C₂₋₁₂ alkynyl which is optionally substituted with one or more substituents selected from halogen, NH₂, N₃, CN, COOH, COO(C₁₋₄ alkyl), OH and C₁₋₄ alkoxy;
   whilst modifications (iii) and (iv) are as defined above.

Where an OH or NAc group on the Le^{a} or Le^{b} oligosaccharide is modified, preferred modifications include replacement of the OH or NAc group with hydrogen, halogen, C₁₋₆ alkoxy, —NR′R″, —NR′COR″, —N(COR′)(COR″), —SR′, —COOR′, —OP(O)(OR′)(OR″), or C₁₋₆ alkyl which is optionally substituted with one or two substituents selected from halogen, NH₂, OH and C₁₋₂ alkoxy, wherein R′ and R″ are identical or different and are selected from hydrogen and C₁₋₆ alkyl groups which are optionally substituted with one or two substituents selected from halogen, NH₂, OH and C₁₋₂ alkoxy. An NAc group may alternatively be replaced with OH. Particularly preferred modifications include replacement of the OH or NAc group with hydrogen, halogen, methoxy, ethoxy, —NH₂, (C₁₋₂alkyl)amine, di(C₁₋₂alkyl)amine, (C₁₋₂acetyl)amine, di(C₁₋₂acetyl)amine, mercapto, methylthio, ethylthio or —OP(O)(OH)₂, or replacement of NAc with OH. Typically, none, one, two or three, preferably none or one, OH or NAc groups in the Le^{a} or Le^{b} oligosaccharide are modified.

Where a hydrogen atom on the Le^{a} or Le^{b} oligosaccharide is modified, preferred modifications include replacement of the hydrogen atom with a halogen, e.g. fluorine.

An alternative modification is the reversal of the axial and equatorial positions on one or more, for example one, carbon atom within the saccharide unit, thus replacement of a hydrogen atom with OH and simultaneous replacement of the OH carried on the same carbon atom with H. If desired, the hydrogen atom may alternatively be replaced with one of the modifications described above for OH groups.

The modifications to hydrogen and OH groups may be made to groups carried on a single carbon atom, resulting in a disubstituted carbon atom. Alternatively, the modifications may be made on different carbon atoms. Where a carbon atom is disubstituted, the OH group is typically modified as described above and the hydrogen atom is typically replaced with OH, halogen, methoxy, ethoxy, —NH₂, (C₁₋₂alkyl)amine, di(C₁₋₂alkyl)amine, (C₁₋₂acetyl)amine, di(C₁₋₂acetyl)amine, mercapto, methylthio, ethylthio or —OP(O)(OH)₂.

Typically, none, one, two or three, preferably none or one, hydrogen atoms in the Le^{a} or Le^{b} oligosaccharide are modified.

Where an —O— moiety on the Le^{a} or Le^{b} oligosaccharide is modified, preferred modifications include replacement of the —O— moiety with —SH—, —NH— or —N(C₁₋₆)alkyl, typically with —SH—, —NH— or —N(Me)-. Typically, none, one, two or three, preferably none or one, —O— groups in the Le^{a} or Le^{b} oligosaccharide are modified.

Thus, preferred Le^{a} and Le^{b} mimetics are those wherein one, two or three, typically one of the following modifications is made:
(i) one or more OH and/or NAc groups are independently replaced with hydrogen, halogen, methoxy, ethoxy, —NH₂, (C₁₋₂alkyl)amine, di(C₁₋₂alkyl)amine, (C₁₋₂acetyl)amine, di(C₁₋₂acetyl)amine, mercapto, methylthio, ethylthio or —OP(O)(OH)₂, or in the case of NAc replaced with OH;
(ii) one or more hydrogen atoms are independently replaced with fluorine;
(iia) the axial and equatorial positions on one or more carbon atoms are reversed;
(iib) one or more carbon atoms are disubstituted such that both the OH and hydrogen are replaced with a group which is independently selected from halogen, methoxy, ethoxy, —NH₂, (C₁₋₂alkyl)amine, di(C₁₋₂alkyl)amine, (C₁₋₂acetyl)amine, di(C₁₋₂acetyl)amine, mercapto, methylthio, ethylthio or —OP(O)(OH)₂;
(iii) one or more —O— moieties are independently replaced with —SH—, —NH— or —N(Me)-;
(iv) in the Glc saccharide unit, the unit —C₁(R)—O— is replaced with —C₁(R)═N—.

In a further preferred embodiment, the Le^{a} or Le^{b} mimetic contains one, two or three modifications (i); and/or one, two or three modifications (ii); and/or one, two or three modifications (iia); and/or one, two or three modifications (iib); and/or one modification (iii); and/or one modification (v).

Typically, the mimetic of Le^{a} or Le^{b} contains a modification (i) and/or modifications (ii).

The structure of Le^{a} and Le^{b} and their mimetics can alternatively be described with reference to formula (I). In formula (I), Y may either represent a group Z (corresponding to Le^{a} and its mimetics) or a saccharide unit of formula (II) (corresponding to Le^{b} and its mimetics). Thus, the compounds of formula (I) can alternatively be depicted as compounds of either formula (Ia) or (Ib):

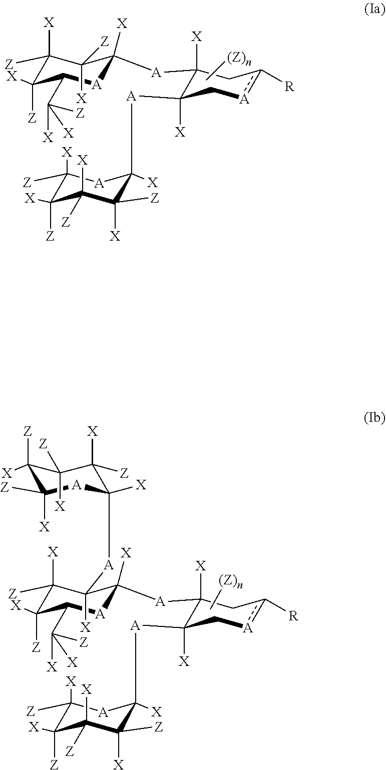

wherein Z, X, n, A and R are as defined for formula (I). For the avoidance of doubt, in formula (Ib) where groups Z, A and X are present on the additional saccharide unit, any group Z within formula (Ib) may be the same or different. Similarly, any group A within formula (Ib) may be the same or different. Further, any group X within formula (Ib) may be the same or different.

In formula (I), (Ia) or (Ib), typically Z is OH, hydrogen, halogen, C₁₋₆ alkoxy, —NR′R″, —NR′COR″, —N(COR′)(COR″), —SR′, —COOR′, —OP(O)(OR′)(OR″), or C₁₋₆ alkyl which is optionally substituted with one or two substituents selected from halogen, NH₂, OH and C₁₋₂ alkoxy, wherein R′ and R″ are identical or different and are selected from hydrogen and C₁₋₆ alkyl groups which are optionally substituted with one or two substituents selected from halogen, NH₂, OH and C₁₋₂ alkoxy. Particularly preferred groups Z are OH, CH₂OH, hydrogen, halogen, methyl, methoxy, ethoxy, —NH₂, (C₁₋₂alkyl)amine, di(C₁₋₂alkyl)amine, (C₁₋₂acetyl)amine, di(C₁₋₂acetyl)amine, mercapto, methylthio, ethylthio or —OP(O)(OH)₂. Most preferred groups Z are OH, CH₂OH, methyl and acetylamine. Typically, either all groups Z are OH, or from 1 to 6, for example one, two, three or four, preferably one or two groups Z are other than OH and the remainder are OH.

X is typically hydrogen or halogen, preferably hydrogen or fluorine.

Alternatively, where a carbon atom carries a group Z which is other than OH, then X on the same carbon atom may represent hydrogen, OH, halogen, C₁₋₆ alkoxy, —NR′R″, —NR′COR″, —N(COR′)(COR″), —SR′, —COOR′, —OP(O)(OR′)(OR″), or C₁₋₆ alkyl which is optionally substituted with one or two substituents selected from halogen, NH₂, OH and C₁₋₂ alkoxy, wherein R′ and R″ are identical or different and are selected from hydrogen and C₁₋₆ alkyl groups which are optionally substituted with one or two substituents selected from halogen, NH₂, OH and C₁₋₂ alkoxy. Particularly preferred groups X in this embodiment are OH, hydrogen, halogen, methoxy, ethoxy, —NH₂, (C₁₋₂alkyl)amine, di(C₁₋₂alkyl)amine, (C₁₋₂acetyl)amine, di(C₁₋₂acetyl)amine, mercapto, methylthio, ethylthio or —OP(O)(OH)₂.

Typically, either each X represents hydrogen, or one, two or three, preferably one, X is other than hydrogen and the remainder represent hydrogen.

Typically A is —O—, —SH—, —NH— or —N(C₁₋₆)alkyl, preferably —O—, —SH—, —NH— or —N(Me)-. Typically, either all groups A are —O— or one, two or three, preferably one, A group is other than —O— and the remainder represent —O—.

n is typically 0, 1, 2 or 3, preferably 2.

As depicted in formula (I), (Ia) and (Ib), the bond C1(R)-A in the terminal saccharide unit corresponding to the Glc unit of Le^{a} or Le^{b}, may be unsaturated or saturated.

Typically, this bond is saturated.

In a preferred embodiment, the drug delivery vehicle of the invention comprises one or more targeting groups comprising an oligosaccharide of formula (Ia) or (Ib) or salt or a PEGylated form thereof:

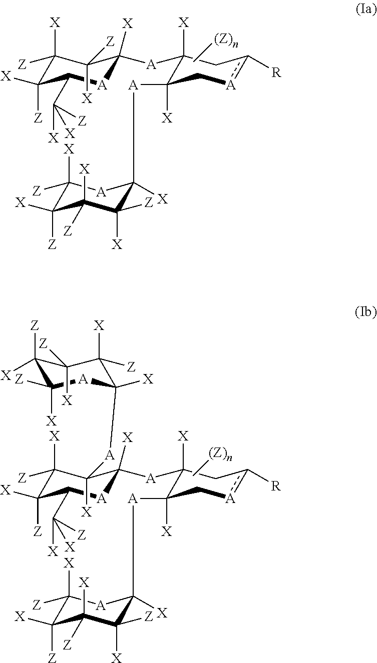

wherein:
each Z is the same or different and is selected from OH, CH₂OH, hydrogen, halogen, methyl, methoxy, ethoxy, —NH₂, (C₁₋₂alkyl)amine, di(C₁₋₂alkyl)amine, (C₁₋₂acetyl)amine, di(C₁₋₂acetyl)amine, mercapto, methylthio, ethylthio and —OP(O)(OH)₂, wherein either all groups Z are OH, or from 1 to 6 groups are other than OH and the remainder are OH; each X is the same or different and is selected from hydrogen or halogen, or in the case where a group Z carried on the same carbon atom as X is other than OH, then X represents OH, hydrogen, halogen, methoxy, ethoxy, —NH₂, (C₁₋₂alkyl)amine, di(C₁₋₂alkyl)amine, (C₁₋₂acetyl)amine, di(C₁₋₂acetyl)amine, mercapto, methylthio, ethylthio or —OP(O)(OH)₂, wherein either all groups X are hydrogen, or one or two groups are other than hydrogen and the remainder are hydrogen;
each A is the same or different and is selected from —O—, —SH—, —NH— and —N(Me)-; wherein either all groups A are —O— or one A group is other than —O— and the remainder represent —O—;
n is 2;
the bond C1(R)-A is saturated; and
R represents the point of attachment to the vesicle.

In a further preferred embodiment,
each Z is the same or different and is selected from OH, CH₂OH, hydrogen, halogen, methyl, methoxy, ethoxy, —NH₂, (C₁₋₂alkyl)amine, di(C₁₋₂alkyl)amine, (C₁₋₂acetyl)amine, di(C₁₋₂acetyl)amine, mercapto, methylthio, ethylthio and —OP(O)(OH)₂, wherein either all groups Z are OH, or from 1 to 6 groups are other than OH and the remainder are OH; each X is the same or different and is selected from hydrogen or halogen, wherein either all groups X are hydrogen, or one or two groups are halogen and the remainder are hydrogen;
each A is —O—;
n is 2;
the bond C1(R)-A is saturated; and
R represents the point of attachment to the vesicle.

Most preferred oligosaccharides of formula (Ia) or (Ib) are Le^{a} and Le^{b}, and their pharmaceutically acceptable salts, in particular Le^{a} or a pharmaceutically acceptable salt thereof.

The Le^{a} or Le^{b} or mimetic thereof may be in the form of a salt. In particular, if a phosphate or sulphonate group is present, this may be in the form of a salt. For in vivo use, the salt will typically be a pharmaceutically acceptable salt. A pharmaceutically acceptable salt is a salt with a pharmaceutically acceptable acid or base. Pharmaceutically acceptable acids include both inorganic acids such as hydrochloric, sulphuric, phosphoric, diphosphoric, hydrobromic or nitric acid and organic acids such as citric, fumaric, maleic, malic, ascorbic, succinic, tartaric, benzoic, acetic, methanesulphonic, ethanesulphonic, benzenesulphonic or p-toluenesulphonic acid. Pharmaceutical acceptable bases include alkali metal (e.g. sodium or potassium) and alkaline earth metal (e.g. calcium or magnesium) hydroxides and organic bases such as alkyl amines, aralkyl amines or heterocyclic amines.

Tautomers of the conjugates of the invention defined above also form part of the invention. Also, conjugates defined above containing one or more chiral centre may be used in enantiomerically or diasteroisomerically pure form, or in the form of a mixture of isomers. The conjugates of the invention typically have more than one chiral centre, which gives rise to diastereoisomers, each of which consists of two enantiomers, with the appropriate (R)- or (S)-stereochemistry at each chiral centre. For the avoidance of doubt, the chemical structures depicted herein are intended to embrace all stereoisomers of the conjugates shown, including racemic and non-racemic mixtures and pure enantiomers and/or diastereoisomers.

For the avoidance of doubt, the conjugates of the invention can, if desired, be used in the form of solvates.

The oligosaccharide may be PEGylated. Standard PEGylation techniques may be used, with the PEG group typically being bound to one of the OH groups on the saccharide. PEG groups of up to 500 units are typically used. Where the oligosaccharide is PEGylated, the PEG group is attached to the oligosaccharide at a position which is not also linked to the vesicle (i.e. the PEG group is not attached at R). The PEGylated oligosaccharide may be an oligosaccharide in salt form.

The oligosaccharide is typically conjugated to a vesicle via the C1 carbon atom of the Glc saccharide unit (for example as depicted at R in formula (I)).

Typically, from 2% to 80% of the lipids making up the vesicle carry an oligosaccharide targeting group, for example, from 5% to 70% or 10% to 60% of lipids. In one embodiment from 20% to 60% of lipids carry an oligosaccharide targeting group.

In addition to oligosaccharide targeting groups, one or more additional targeting groups may be conjugated to the vesicle. For instance, further conjugating groups considered to aid transport across the blood brain barrier may be used. Such targeting groups include glutathione, transferrin, and antibodies that bind to transferrin receptors, for example.

### Vesicles

Vesicles are known to be useful in the delivery of drugs to a subject. Suitable active therapeutic agents can be encapsulated within the vesicle and delivered to the target site where they release their drug payload. Vesicles suitable for use in the invention include endogenous or naturally occurring vesicles such as exosomes, microvesicles or apoptotic bodies. Synthetic vesicles, also known as liposomes, can also be used. Liposomes or exosomes, in particular liposomes, are preferred.

Liposomes suitable for use in drug delivery are described, for example, by Akbarzadeh et al (Nanoscale Res Lett, 2013; 8(1):102 and the liposomes discussed therein are incorporated herein by reference. Liposomes vary from very small molecules of 60 nm or less in size, up to much larger molecules of over 1 um. Vesicles (e.g. liposomes) of at least 100 nm are preferred for use in the present invention. Vesicles having this size have a greater capacity to carry therapeutic agent. Further, vesicles having a smaller size have weaker binding to cell adhesion molecules. In addition, vesicles should not be overly large as this may inhibit their ability to cross the BBB. Vesicles therefore preferably have a particle size of from 100 nm to 1 um, more preferably from 100 to 800 nm, preferably 100 to 500 nm. The particle size is most preferably at least 200 nm, for example from 200 nm to 800 nm, e.g. 200 to 500 nm, e.g. about 300 nm.

Preferred vesicles for use in the present invention are liposomes, in particular liposomes having a particle size of from 100 nm to 1 um, preferably from 100 nm to 800 nm, e.g. 100 to 500 nm, e.g. 200 nm to 800 nm or 200 to 500 nm.

Liposomes can be synthesised by techniques known to the skilled person in the art, such as those described by Akbarzadeh (referenced above). Similarly, exozomes or other endogenous vesicles can be isolated by known techniques.

Typically, about 5 to 80% of lipids in the vesicle are conjugated to targeting groups. For instance, from 10 to 70%, e.g. from 20 to 60% of lipid molecules may carry a targeting group.

### Therapeutic Agents

The therapeutic agent which may be carried by the drug delivery vehicle of the invention is not particularly limited and may include biologic molecules, for example antibodies, siRNA, mRNA, proteins and lipids. Small molecule drugs may also be carried by the vesicle. Two or more different therapeutic agents may be encapsulated into the vesicle, and pharmaceutical compositions may comprise two or more vesicles, each containing different therapeutic agents.

Therapeutic agents which are useful in the present invention are those which are known to prevent or treat diseases or disorders of the brain, in particular those associated with inflammation where targeting activated endothelium and tissue surrounding activated endothelium is useful (for example microglial cells or other brain parenchymal cells in areas of inflammation). Thus, the therapeutic agent may be one known to be useful in the prevention or treatment of Alzheimer's disease, progressive supranuclear palsy, frontotemporal dementia, multiple sclerosis, metastasis, spinal cord injury and stroke. In particular, therapeutic agents useful in the treatment of Alzheimer's disease, multiple sclerosis and metastasis may be used.

In one embodiment, the therapeutic agent is an inhibitor of deubiquiinating enzyme (DUB). Such inhibitors are thought to be useful in the treatment of diseases such as Alzheimer's disease, but selectively delivering these drugs to the brain remains problematic. The present invention provides a suitable means to deliver these agents to the sites of interest. One example of a DUB inhibitor is VLX1570. The vesicle may therefore contain a DUB inhibitor, preferably VLX1570. A combination of two or more DUB inhibitors may be used and one or more further therapeutic agents may optionally be present. The therapeutic agent may be encapsulated within the vesicle by conventional drug loading techniques known to the skilled person in the art. For instance, drug loading may be passive, i.e. the drug may be encapsulated during liposome formation, or active, i.e. after liposome formation.

### Production of Drug Delivery Vehicles

Le^{a} and Le^{b} can be prepared as set out in Schemes 1a and b and 2a and b below:

### Synthesis of Lewis a

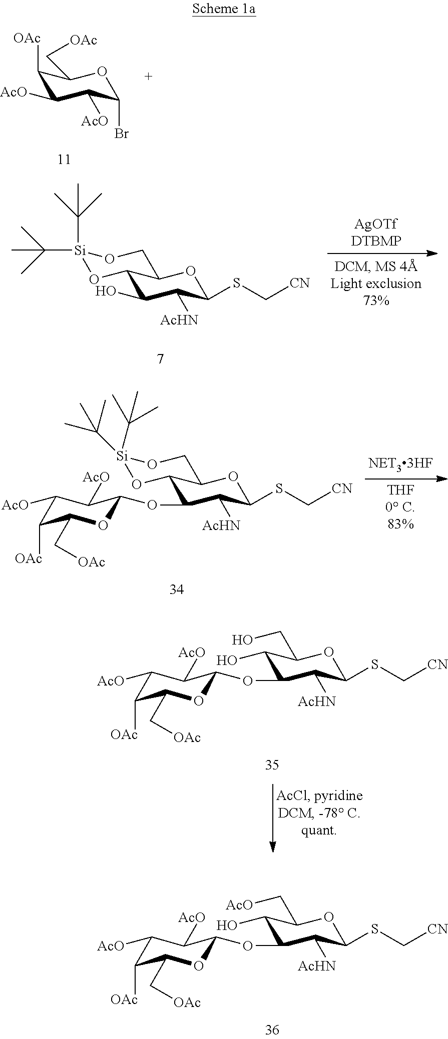

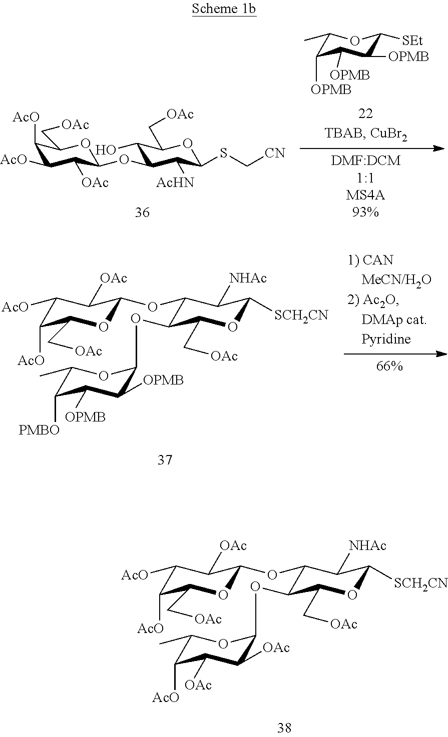

### Synthesis of Lewis b

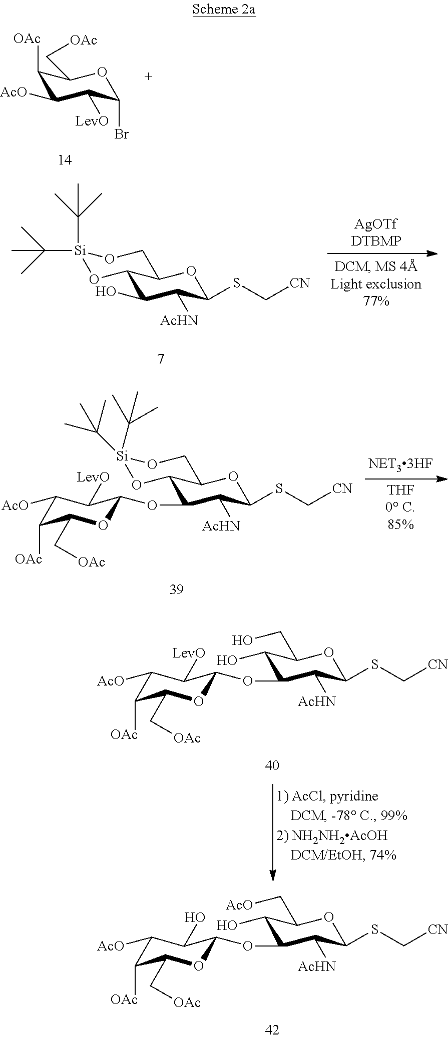

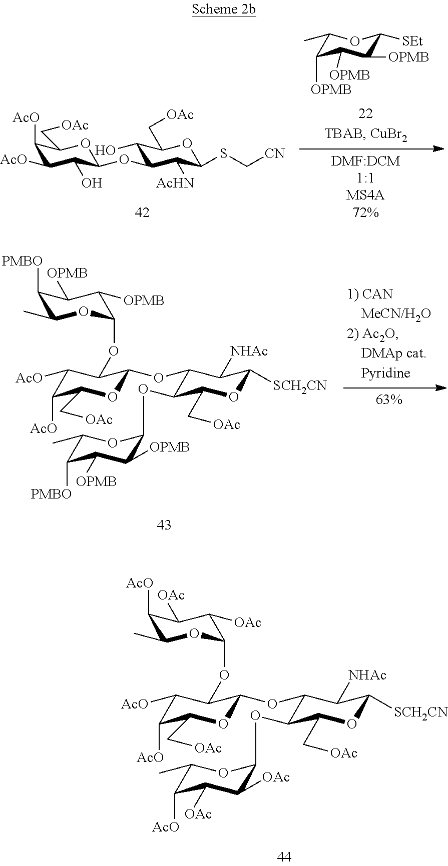

The AcO groups can be removed by standard deprotection techniques, typically after conjugation of the oligosaccharide to a lipid. Further detail regarding the synthetic route to Lewis A and Lewis B is provided in the Examples.

The mimetics of Le^{a} and Le^{b} can be prepared by adaptation of the above Schemes in an appropriate way, as would be familiar to the skilled chemist. For instance, the GluNAc moiety could be replaced by a cyclohexyl ring substituted by different substituents to graft the Gal and Fuc moieties. Cyclohexanediol can be used to synthesize this type of mimetic. The O-glycosidic linkages could be switch to C—, S— or amide glycosidic linkages. The hydroxyl groups could be also modified by different substituents using previously modified monosaccharide units.

Schemes 1 and 2 above provide the oligosaccharide with a reactive —SCH₂CN group at the position which is to conjugate with the vesicle. Thus, a lipid group derivatised with an amine group, for example, can be reacted with the oligosaccharide, providing an oligosaccharide with a lipid tail. Lewis A and Lewis B conjugated to lipid groups are also commercially available.

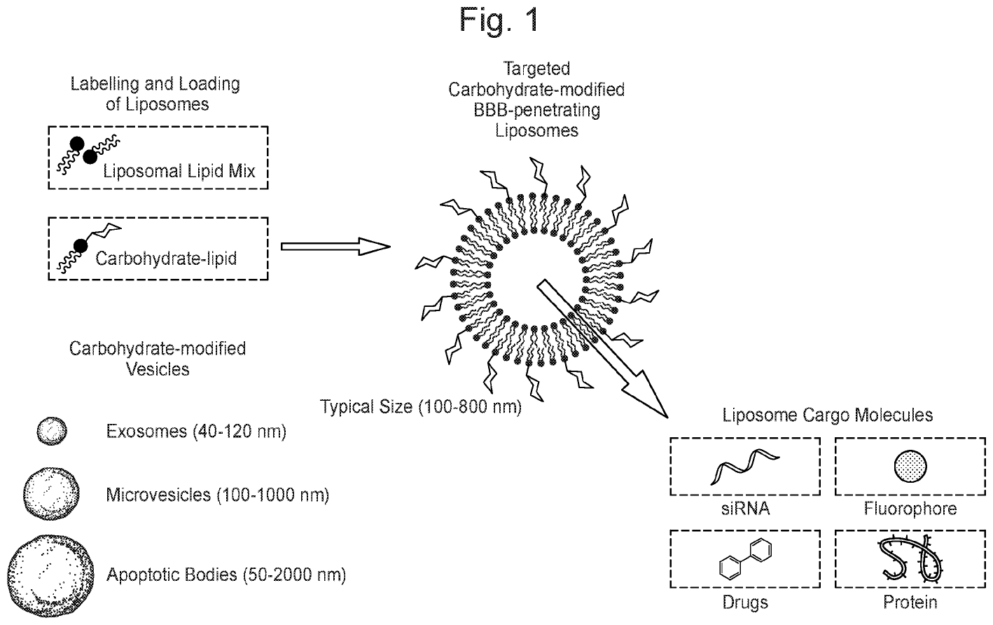

The oligosaccharide-lipid conjugate can be incorporated into a vesicle. For example, addition of the conjugate during initial formation of the liposome provides a liposome incorporating oligosaccharide targeting groups. Such a route is schematically depicted in FIG. 1. Alternatively, vesicles can be incubated in the presence of the lipid-oligosaccharide conjugate leading to incorporation of the conjugate into the vesicle. Typically, the oligosaccharide-lipid conjugate is combined with non-targeted lipids in a molar ratio of from 2:98 to 80:20 (oligosaccharide-lipid:non-targeted lipid), for instance in a molar ratio of 5:95 to 70:30, preferably in a molar ratio of 10:90 to 60:40, e.g. 20:80 to 60:40. This provides a content of oligosaccharide-targeted lipid of from 2 to 80%, preferably from 5 to 70%, e.g. about 10% to about 60% by mole, e.g. from about 20% to about 60% by mole.

### Pharmaceutical Compositions

The drug delivery vehicles of the invention can be formulated for use by combination, in a pharmaceutical composition, with a pharmaceutically acceptable carrier or diluent. The compositions are typically prepared following conventional methods and are administered in a pharmaceutically suitable form.

Solid oral forms of the composition of the invention may contain, together with the conjugated particles themselves, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non toxic and pharmacologically inactive substances used in pharmaceutical formulations. Such compositions may be manufactured in known manner, for example, by means of mixing, granulating, tableting, sugar coating, or film coating processes.

Liquid dispersions for oral administration may be syrups, emulsions and suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspension or solutions for intramuscular injections may contain, together with the conjugated particles of the invention, a pharmaceutically acceptable carrier, e.g. sterile water, olive oil, ethyl oleate, glycols, e.g. propylene glycol, and if desired, a suitable amount of lidocaine hydrochloride.

Solutions for injection or infusion may contain as carrier, for example, sterile water or preferably they may be in the form of sterile, aqueous, isotonic saline solutions. Most preferably the composition comprises a drug delivery vehicle according to the invention and saline.

### Therapeutic Uses

The drug delivery vehicles of the invention are typically formulated for parenteral administration, for example for intravenous injection. In one embodiment the drug delivery vehicles or compositions are injected into the peripheral blood. The dosages in which the drug delivery vehicles according to the invention are administered, and the drug loading of the drug delivery vehicle, will vary according to the mode of use, nature of the active agent, as well as the requirements of the subject. Typically, the drug delivery vehicles of the invention will be administered, and drug loading controlled, so as to deliver an equivalent or lower amount of drug, compared to delivery of the same active therapeutic agent by other means. Lower dosages, whilst achieving equivalent activity, may be administered due to the improved targeting of the therapeutic agent to the desired site.

The drug delivery vehicles may be administered in a dosage, and with a drug loading, which is selected according to the nature and severity of the disease or condition to be treated and factors connected with the patient, in particular the age and body weight, as well as other relevant factors. The dosage given is typically in the range of from 1 mg to 3 g per day.

The drug delivery vehicles of the invention are advantageous since they selectively target cell adhesion molecules, which then carry the drug delivery vehicles with their drug payload across the blood brain barrier and into brain cells. They are therefore able to selectively target activated endothelium and brain parenchymal cells such as microglia. Microglial cells are associated with tau pathology and selective targeting of these cells is therefore of value in the treatment of diseases in which tau pathology is implicated. This includes Alzheimer's Disease, progressive supranuclear palsy and frontotemporal dementia.

More generally, the drug delivery vehicles are useful in the delivery of drugs to activated endothelium and microglial cells. They are therefore useful in the treatment and prevention of diseases and disorders of the brain. Particular diseases and disorders which the drug delivery vehicles and compositions may be used to treat or prevent therefore include Alzheimer's disease, progressive supranuclear palsy, frontotemporal dementia, multiple sclerosis, metastasis, spinal cord injury and stroke. In particular, the drug delivery vehicles and compositions are useful in treatments for, or prevention of, Alzheimer's disease, multiple sclerosis and metastasis. Prevention may include prophylactic treatment. Treatment may include cure of the disease, treatment of symptoms of the disease, and reducing progression of disease.

The invention will now be described with reference to the following Examples. Reference numerals in Examples 1 and 2 refer to Schemes 1a, 1b, 2a and 2b above.

### EXAMPLES

### Example 1: Synthesis of Lewis A

Step 1a: 2-N-Acetamido-3,4,6-tri-O-acetyl-2-deoxy-1-chloro-β-D-glucopyranoside 2

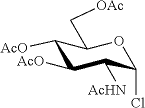

Acetyl chloride (58.1 mL, 831.6 mmol, 9 eq.) was placed in a 250 mL round-bottom flask and N-Acetyl glucosamine 1 (20.0 g, 90.4 mmol, 1.0 eq.) was added within 3 min under strong stirring and Ar-atmosphere. The suspension is stirred for 2 days at 25° C. before CHCl₃ (200 mL) was added to the slightly discoloured solution. The mixture is poured under strong stirring onto ice (200 g) and H₂O (50 mL). Phases were separated and the organic layer transferred without delay into a 1 L beaker with ice (100 g) and satd. NaHCO₃-solution (250 mL). The mixture was shortly stirred, transferred to a separatory funnel and shaken until the gas-production ended. Phases were separated, MgSO₄ (15 g) was added to the organic layer, stirred for no longer than 10 min, filtrated and the solvents concentrated under vacuum to a volume of about 20 mL before Et₂O (250 mL) was rapidly added and the solution allowed to crystallize at 25° C. Formed crystals are filtered off (22.5103 g) and the mother liquor evaporated to give a syrup, which on addition of 30 mL Et₂O yielded more of the chloride (1.3437 g) to yield 2 as colourless crystals (23.854 g, 65.22 mmol, 72%). TLC: R_{f}=0.47 (EtOAc neat).

### Step 1b: S-thiouronium 2-N-Acetamido-3,4,6-tri-O-acetyl-2-deoxy-1-thio-β-D-glucopyranoside 3

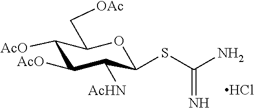

Crude chloride 2 (23.854 g, 65.22 mmol, 1.0 eq.) was dissolved in dry acetone (200 mL). Thiourea (9.93 g, 130.44 mmol, 2 eq.) was added, and the mixture heated to reflux (80° C.). After 2 h the solution is cooled to 25° C., filtrated and the solids washed with ice-cold dry EtOH (30 ml). The filtrate is concentrated to dryness, dissolved in acetone under heat and allowed to crystallize at 25° C. Filtration, washing, and drying of the combined solid fractions gave 3 as white powder (23.3931 g, 52.94 mmol, 81%). TLC: R_{f}=0.2 (MeOH neat).

### Step 1c: S-cyanomethyl 2-N-Acetamido-3,4,6-tri-O-acetyl-2-deoxy-1-thio-β-D-glucopyranoside 4

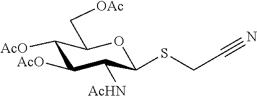

### S-thiouronium 2-N-Acetamido-3,4,6-tri-O-acetyl-2-deoxy-1-thio-β-D-glucopyranoside 3

(23.3 g, 52.7 mmol, 1.0 eq.) was dissolved in a water/acetone mixture (1:1, 240 mL) and sodium metabisulphite (22.0 g, 115.9 mmol, 2.2 eq.), potassium carbonate (9.5 g, 68.5 mmol, 1.3 eq.) and chloroacetonitrile (63.4 mL, 1.0 mol, 19 eq.) were added. After 16 h at 25° C. the solution was poured on ice-water (500 mL) and stirred for another 1 h. The mixture was transferred to a separatory funnel and the organic material extracted with CH₂Cl₂ (3×250 mL). The combined organic layers were washed with ice-cold brine (2×250 mL), filtered through cotton, solvents evaporated, and twice codestilled with acetone. The title compound 4 was obtained as white powder (24.6 g, 61.0 mmol, quant.) which was used without further purification in the next reaction. TLC: R_{f}=0.60 (EtOAc:MeOH, 8:2).

### Steph 1d: S-cyanomethyl 2-N-Acetamido-2-deoxy-1-thio-β-D-glucopyranoside 5

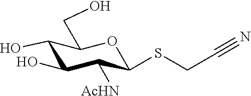

To a suspension of tetraacetate 4 (17.3 g, 43.0 mmol, 1.0 eq.) in MeOH (340 mL), Et₃N (34.0 mL, 240.7 mmol, 5.6 eq.) was added and stirred at r.t. for 24 h. Solvents were removed under high vacuum and the crude mixture was coevaporated with toluene (2×20 mL) to afford 5 as a white powder (12.7 g, 45.9 mmol, quant.) which was used for the next step without further purifications. TLC: R_{f}=0.14 (EtOAc:MeOH, 8:2).

### Step 1e: S-cyanomethyl 2-N-Acetamido-4,6-O-di-tert-butylsilylidene-2-deoxy-1-thio-β-D-glucopyranoside 7

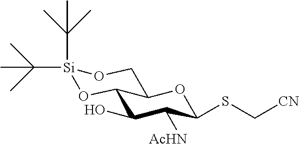

To 5 (5.0 g, 17.9 mmol, 1 eq) in anhydrous degaz DMF (80 mL) at −40° C. (Dry ice/MeCN) was slowly added di-tert-butylsilyl bis(trifluomethanesulfonate) (6.2 mL, 18.8 mmol, 1.05 eq). The mixture was stirred overnight from −40° C. to 0° C. under N2. Pyridine (5 mL) was then added and stirred for 10 min at 4° C. to quench the reaction before evaporation under vacuum. The crude was co-evaporated 3 times with toluene before purification by flash chromatography over silica (biotage gradient DCM 0% to 5% MeOH in DCM follow by an isocratic gradient of 15% MeOH in DCM) to afford 7 as a white anamorphous powder (5.7 g, 21.1 mmol, 76%).

### Step 1f: 1-bromo-2,3,4,6-tetra-O-acetyl-D-galactopyranose 11

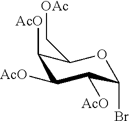

1,2,3,4,6-penta-O-acetyl-β-D-galactopyranose (20.00 g, 57.47 mmol) was dissolved in CH₂Cl₂ (123 mL) and HBr in HOAc (33% v/v, 28 mL). The mixture was stirred for 2 h at room temperature. Then the organic phase was washed with saturated aqueous NaHCO₃ (1×100 mL, 2×50 mL) and brine (1×10 mL). After drying (MgSO₄) and concentration, 1-bromo-2,3,4,6-tetra-O-acetyl-D-galactopyranose 11 (16.75 g, 40.73 mmol, 71%) was obtained as a white amorphous powder (stored at −20° C.) and was used for the next step without further purification.

### Step 1g: S-ethyl 2,3,4-tri-O-p-methoxybenzyl-6-deoxy-1-thio-β-L-galactopyranoside 22

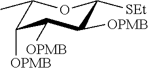

To a solution of L-Fucose (25.0 g, 152.3 mmol, 1.0 eq) in dry pyridine (200.0 mL) was added acetic anhydride (130 mL, 1307.7 mmol, 9.0 eq). The reaction was stirred at rt ON. Solvent were evaporated under high vacuum and the crude mixture was co-evaporated 3 times with toluene to afford 18 (TLC: R_{f}=0.50, EtOAc:PE, 8:2) which was directly diluted in DCM (600 mL) under argon. Hydrogen bromide 33% in acetic acid (330 mL, 1828.0 mmol, 12 eq) was added ad the reaction was stirred at rt for 2 h. The crude mixture was poured into a mixture of water and ice (1 L) and extract with DCM (2×500 mL). The combined organic layers were washed with water (500 mL), NaHCO₃ sat. (500 mL) and brine (500 mL), dried over MgSO₄, filtered and concentrated in vacuo. The crude 19 was then dissolved in acetone (440 mL) and EtOAc (22 mL). Ethyl mercaptan (13 mL, 175.1 mmol, 1.15 eq) was then added follow by KOH (96.2 g, 175.1 mmol, 1.15 eq) in EtOH (92.2 mL). The solution was stirred at rt for 16 h and diluted in 500 mL DCM. The solution was washed with NaHCO₃ sat. (600 mL) and brine (600 mL), dried over MgSO₄, filtered and concentrated under vacuo to afford the crude 20, which was used as it for the next step.

To the crude 20 dissolved in dry MeOH (150 mL) was added sodium methoxide (9.5 g, 175.1 mmol, 1.15 eq). The solution was stirred at rt for 1 h before neutralization with Dowex H+ form and filtered. The crude mixture and tetra-butylammonium iodide (5.6 g, 15.23 mmol, 0.1 eq) in dry DMF (610 mL) were cooled at 0° C. before sodium hydride 60% in oil suspension (37 g, 913.8 mmoL, 6.0 eq) was added. The solution was stirred at 0° C. 30 min and paramethoxybenzyl chloride (83 mL, 609.2 mmol, 4 eq) was added. The solution was allowed to warm to r.t. and stirred 22 h. Methanol was added at 0° C. to quenched the reaction and the solution was concentrated in vacuo, diluated in EtOAc (500 mL) and washed with Brine (4×400 mL), dried over MgSO4, filtered and re-concentrated in vacuo before purification by flash chromatography over silica (PE: EtOAc, 9:1, 85:15, 8:2) which provide 22 as slightly yellow powder (43 g, 75.6 mmol, 50%). TLC: R_{f}=0.66 (EtOAc neat).

### Step 1h: S-cyanomethyl 2-N-acetimido-4,6-O-di-tert-butylsilylidene-3-O-[2,3,4,6-tetra-O-acetyl-β-D-galactopyranosyl]-2-deoxy-1-thio-β-D-glucopyranoside 34

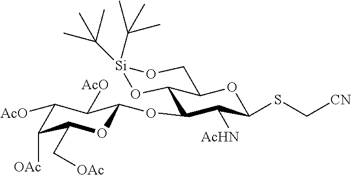

Acceptor 7 (1.0 g, 2.4 mmol, 1 eq) and donor 11 (1.5 g, 3.6 mmol, 1.5 eq) were co-evaporated twice with toluene and dry under high vacuum for 1 h. DCM (24 mL) and MS 4 Å (2.4 g) were then added and the solution was stirred at r.t. for 1 h before addition of DTBMP (493 mg, 2.4 mmol, 1 eq) and AgOTf (1.4 g, 5.5 mmol, 2.3 eq). The mixture was covered by aluminium foil and stirred at r.t. After 2.5 h the reaction mixture was filter over Celite and rinse with DCM. After removal of the solvent, the residue was purified by flash chromatography on silica (biotage gradient 12% to 100% EtOAc in PE) to afford 34 as a white anamorphous solid (1.3 g, 1.7 mmol, 71%).

### Step 1i: S-cyanomethyl 2-N-acetimido-3-O-[2,3,4,6-tetra-O-acetyl-β-D-galactopyranosyl]-2-deoxy-1-thio-β-D-glucopyranoside 35

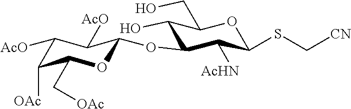

To protected 34 (1.2 g, 1.6 mmol, 1 eq) in dry THF (16 mL) at 0° C. was added NEt₃·3HF (530 μL, 3.3 mmol, 2.1 eq). After 3 h solvent were evaporated. The crude residue was purified by flash chromatography over silica (biotage gradient, 0% to 5% MeOH in EtOAc) to afford 35 as a white anamorphous solid (777.3 mg, 1.3 mmol, 87%).

### Step 1j: S-cyanomethyl 2-N-acetimido-6-O-Acetyl-3-O-[2,3,4,6-tetra-O-acetyl-β-D-galactopyranosyl]-2-deoxy-1-thio-β-D-glucopyranoside 36

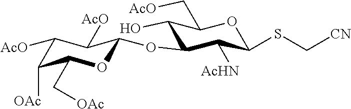

To unprotected disaccharide 35 (735.0 mg, 1.2 mmol, 1 eq) in dry DCM (17 mL) and dry pyridine (360 μL, 4.4 mmol, 4 eq) under argon at −78° C. was added AcCl (200 μL, 2.7 mmol, 2.5 eq). After 1 h methanol was added to quench the reaction and stirred a bit at −78° C. The crude was dissolve in DCM (200 mL) and washed with HCl 1 N (100 mL), NaHCO₃ (100 mL), Brine (100 mL) and dried over MgSO₄. Evaporation of the solvent to dryness afford 36 as a white anamorphous solid (801.0 mg, 1.2 mmol, quant.)

### Step 1k: S-cyanomethyl 2-N-acetamido-6-O-acetyl-4-O-[2,3,4-tri-O-p-methoxybenzyl-6-deoxy-α-L-galactopyranosyl]-3-O-[2,3,4,6-tetra-O-acetyl-β-D-galactopyranosyl]-2-deoxy-1-thio-β-D-glucopyranoside 37

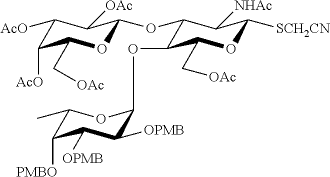

36 (1.4 g, 2.2 mmol, 1.0 eq) and 22 (2.5 g, 4.4 mmol, 2.0 eq) (co-evaporated 3× with toluene and dried under high vacuum for 1 h) were dissolved in dry DCM:DMF 1:1 mixture (4.4 mL) and MS 4 Å (2.2 g) and stirred 1 h at rt before copper(II) bromide (987.0 mg, 4.4 mmol, 2.0 eq) and tetra-butylammonium bromide (1.5 g, 4.6 mmol, 2.1 eq) were added. The mixture was stirred at rt overnigth with light exclusion. The crude mixture was filtered over celite and washed with EtOAc (400 mL). The filtrate was washed with NH₄Cl sat pH 8.5 aqueous solution (3×200 mL) and brine (200 mL). The organic layers was dried over MgSO₄, filtered, concentrated in vacuum and purified by flash chromatography (biotage gradient, 50% to 100% EtOAc in PE) to afford 37 as a white cream foam (2.3 g, 2.0 mol, 91%).

### Step 1l: S-cyanomethyl 2-N-acetamido-6-O-acetyl-4-O-[2,3,4-tri-O-acetyl-6-deoxy-β-L-galactopyranosyl]-3-O-[2,3,4,6-tetra-O-acetyl-β-D-galactopyranosyl]-2-deoxy-1-thio-β-D-glucopyranoside 38

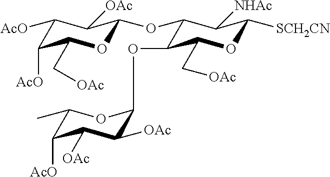

To 37 (2.2 g, 1.9 mmol, 1.0 eq) dissolved in a mixture of MeCN:H₂O (9:1, 39.0 mL) was added CAN (6.9 g, 12.6 mmol, 6.5 eq). The solution was stirred at r.t. for 7 h before acetic anhydride (165 mL), Pyridine (165 mL) and DMAP (24 mg, 0.2 mmol, 0.1 eq) were added. The mixture was stirred ON at r.t. before MeOH was added to quench the reaction. Solvant (with co-distillation with toluene 3 times) and the crude mixture was diluted in EtOAc (500 mL). The organic layer was washed with HCl 1M (3×200 mL), NaHCO₃ sat. aqueous solution (3×200 mL), Brine (200 mL, dried over MgSO₄, filtered and concentrated under vacuo. The crude mixture was then purified by flash chromatography over silica (biotage, 70% to 100% EtOAc in PE) follow by a water wash afford 38 a white anamorphous solid (1.2 g, 1.3 mmol, 68%).

### Step 1m: S-cyanomethyl 2-N-acetamido-4-O-[6-deoxy-α-L-galactopyranosyl]-3-O—[β-D-galactopyranosyl]-2-deoxy-1-thio-β-D-glucopyranoside 23

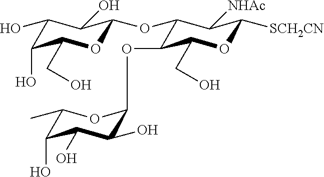

Acylated modified Le^{a} 38 (10.0 mg, 10.9 μmol) was disolved in MeOH (109 μL). MeONa (25% solution, 2.483 L) was added and the mixture was stirred for 5 min at room temperature. The reaction was quenched by introduction of ion exchange resin (Dowex, 100-200 mesh). After filtration and concentration (N₂ flow), the modified Le^{a} 23 (8.4 mg, 9.2 μmol, 84%) was isolated as an amorphous white powder. ¹H NMR (500 MHz, MeOD) δ 5.05 (d, J=3.9 Hz, 1H, H″-1), 4.88 (dq, J=6.7, 0.6 Hz, 1H, H″-5), 4.72 (br d, J=9.9 Hz, 1H, H-1), 4.44 (d, J=7.6 Hz, 1H, H′-1), 4.06 (m, 1H, H-2), 4.01 (m, 1H, H-3), 3.96 (dd, J=12.4, 2.3 Hz, 1H, H-6a), 3.92 (dd, J=12.4, 3.5 Hz, 1H, H-6b), 3.86 (dd, J=10.2, 3.3 Hz, 1H, H″-3), 3.85 (d, J=17.1 Hz, 1H, SCHCN), 3.84-3.80 (m, 1H, H-4), 3.76 (dd, J=10.9, 4.0 Hz, 1H, H′-6a), 3.76 (dd, J=10.2, 4.0 Hz, 1H, H″-2), 3.74 (dd, J=3.4, 0.6 Hz, 1H, H″-4), 3.74 (dd, J=3.3, 0.8 Hz, 1H, H′-4), 3.69 (dd, J=11.4, 4.9 Hz, 1H, H′-6b), 3.63 (d, J=17.0 Hz, 1H, SCHCN), 3.52 (dd, J=9.5, 7.6 Hz, 1H, H′-2), 3.47 (ddd, J=9.7, 3.5, 2.4 Hz, 1H, H-5), 3.43 (ddd, J=6.9, 4.9, 0.8 Hz, 1H, H′-5), 3.42 (dd, J=9.6, 3.3 Hz, 1H, H′-3), 1.99 (s, 3H, NHAc), 1.20 (d, J=6.6 Hz, 3H, H″-6). ¹³C NMR (126 MHz, MeOD) δ 174.0 (NHC(═O)CH₃), 118.5 (CN), 105.1 (C′-1), 99.7 (C″-1), 84.7 (C-1), 82.1 (C-5), 79.6 (C-3), 76.8 (C′-5), 74.8 (C′-3), 73.7 (C″-4), 73.4 (C-4), 72.2 (C′-2), 71.2 (C″-3), 70.0 (C″-2), 69.8 (C′-4), 67.7 (C″-5), 62.9 (C′-6), 61.4 (C-6), 55.8 (C-2), 23.0 (NHC(═O)CH₃), 16.6 (H″-6), 14.9 (SCH₂CN). HRMS (m/z): calcd for C₂₂H₃₆N₂NaO₁₄S, 607.1779 ([M+Na]⁺). Found 607.1781.

### Step 1n: Mixture of S-cyanomethyl 2-N-acetamido-4-O-[6-deoxy-α-L-galactopyranosyl]-3-O—[β-D-galactopyranosyl]-2-deoxy-1-thio-β-D-glucopyranoside 23 and S-2-imino-2-methoxyethyl 2-N-acetamido-4-O-[6-deoxy-α-L-galactopyranosyl]-3-O—[β-D-galactopyranosyl]-2-deoxy-1-thio-β-D-glucopyranoside 45

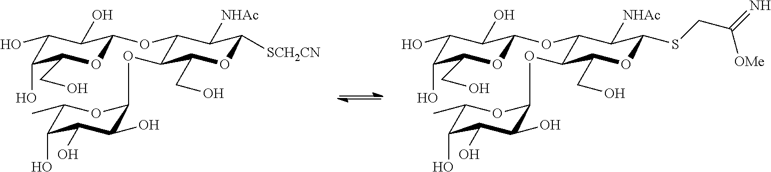

Acylated modified Le^{a} 38 (51.4 mg, 55.9 μmol) was dissolved in anhydrous MeOH (598.3 μL) under argon. NaOMe (1M, 22.34 μL, 0.4 equiv) was added and the mixture was stirred for 24 h at room temperature. After concentration (N₂ flow for 30 min and high vacuum for 1 h), a mixture of the activated oligosaccharide 4-O-[6-deoxy-α-L-galactopyranosyl]-3-O—[β-D-galactopyranosyl]-1-(1-thio-S-2-imino-2-methoxyethyl)-2-acetamido-2-deoxy-β-D-glucopyranoside 45 and of the non-activated oligosaccharide 4-O-[6-deoxy-α-L-galactopyranosyl]-3-O—[β-D-galactopyranosyl]-1-thio-S-cyanomethyl-2-acetamido-2-deoxy-β-D-glucopyranoside 23 was obtained (36.0 mg, 51.7 mol % and 53.0 mass % of 45).

### Example 2: Synthesis of Lewis B

### Step 2a: 1,3,4,6-tetra-O-acetyl-α-D-galactopyranose 12

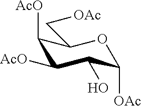

1,2,3,4,6-penta-O-acetyl-β-D-galactopyranose (50.03 g, 128.2 mmol) was dissolved in TFA/H₂O 10:1 (193 mL). The mixture was stirred for 5 h at room temperature. Then the EtOAc was evaporated and the residue was co-evaporated with toluene (3*30 mL) before dilution in diisopropylether (500 mL) to concretize a precipitate. After 45 min stirring at rt, 1,3,4,6-tetra-O-acetyl-α-D-galactopyranose 12 (21.16 g, 60.74 mmol, 47%) was isolated as a white amorphous solid by filtration and wash (3*150 mL diisopropylether). The compound was used for the next step without further purification.

### Step 2b: 1,3,4,6-tetra-O-acetyl-2-O-levulinoyl-α-D-galactopyranose 13

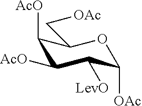

1,3,4,6-tetra-O-acetyl-D-galactopyranose 12 (17.00 g, 48.81 mmol) and levunilic acid (6.91 g, 6.94 mmol, 1.2 equiv) were dissolved in anhydrous DCM (195 mL). N-(3-Dimethylaminopropyl)-N′-ethylcarbodiimide hydrochloride (18.76 g, 95.90 mmol, 2.0 equiv), triethylamine (6.81 mL, 48.8 mmol, 1.0 equiv) and dimethylaminopyridine (1.19 g, 9.76 mmol, 0.2 equiv) were introduced and the mixture was stirred at room temperature for 5 h. The mixture was washed with water (1×250 mL), HCl 1N (2×250 mL), NaHCO₃ (1×300 mL) and brine before drying over MgSO₄ and concentration. 1,3,4,6-tetra-O-acetyl-2-O-levulinoyl-□-D-galactopyranose 13 (20.27 g, 45.41 mmol, 93%) was isolated as a translucent oil and was used for the next step without further purification.

### Step 2c: 3,4,6-tri-O-acetyl-2-O-levulinoyl-1-bromo-α-D-galactopyranose 14

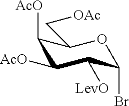

1,3,4,6-tetra-O-acetyl-2-O-levulinoyl-β-D-galactopyranose 13 (19.0 g, 42.5 mmol) was dissolved in hydrobromic acid 33% wt. in acetic acid solution (42.0 mL, 1.0 mL/mmol, 240.0 mmol, 5.6 equiv) and acetic anhydride (6.0 mL, 0.14 mL/mmol, 63.5 mmol, 1.5 equiv). The mixture was stirred at room temperature for 5 hours. The mixture was directly extracted with EtOAc (500 mL) and the organic layer was washed with water (250 mL), NaHCO₃ saturated solution (3×250 mL) and brine (150 mL). After drying over MgSO₄ and concentration, the titled compound 14 (19.0 g, 40.7 mmol, 96%) was isolated as a yellow oil and was used for the next step without further purification.

### Step 2d: S-cyanomethyl 2-N-acetimido-4,6-O-di-tert-butylsilylidene-3-O-[2-O-levulinoyl-3,4,6-tri-O-acetyl-β-D-galactopyranosyl]-2-deoxy-1-thio-β-D-glucopyranoside 39

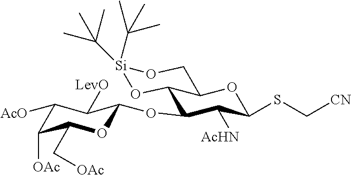

Acceptor 7 (Prepared as described in Example 1; 100 mg, 240 μmol, 1 eq) and donor 14 (168 mg, 360 μmol, 1.5 eq) were co-evaporated twice with toluene and dry under high vacuum for 1 h. DCM (2.4 mL) and MS 4 Å (240 mg) were then added and the solution was stirred at r.t. for 1 h before addition of DTBMP (49 mg, 240 μmol, 1 eq) and AgOTf (142 mg, 552 μmol, 2.3 eq). The mixture was covered by aluminium foil and stirred at r.t. After 2.5 h the reaction mixture was filter over Celite and rinse with DCM. After removal of the solvent, the residue was purified by flash chromatography on silica (biotage gradient 0% to 10% EtOH in CHCl₃) and resubject to a second purification by flash chromatography on (biotage gradient 50% to 100% EtOAc in PE) to afford 39 as a white anamorphous solid (135 mg, 168 μmol, 70%).

### Step 2e: S-cyanomethyl 2-N-acetimido-3-O-[2-O-levulinoyl-3,4,6-tri-O-acetyl-β-D-galactopyranosyl]-2-deoxy-1-thio-β-D-glucopyranoside 40

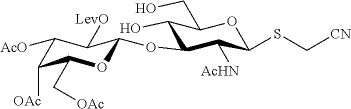

To protected 39 (1.5 g, 1.8 mmol, 1 eq) in dry THF (18 mL) at 0° C. was added NEt₃·3HF 6530 μL, 3.8 mmol, 2.1 eq). After 1.5 h solvent were evaporated. The crude residue was purified by flash chromatography over silica (biotage gradient, 0% to 10% MeOH in EtOAc) to afford 40 as a white anamorphous solid 867.7 mg, 1.3 mmol, 72%)

### Step 2f: S-cyanomethyl 2-N-acetimido-6-O-Acetyl-3-O-[2-O-levulinoyl-3,4,6-tri-O-acetyl-β-D-galactopyranosyl]-2-deoxy-1-thio-β-D-glucopyranoside 41

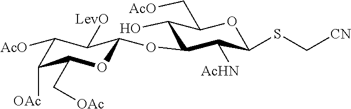

To unprotected disaccharide 40 (700 mg, 1.1 mmol, 1 eq) in dry DCM (16 mL) and dry pyridine (340 μL, 4.2 mmol, 4 eq) under argon at −78° C. was added AcCl (190 μL, 2.6 mmol, 3 eq). After 1 h methanol was added to quench the reaction and stirred a bit at −78° C. The crude was dissolve in DCM (200 mL) and washed with HCl 1 N (100 mL), NaHCO₃ (100 mL), Brine (100 mL) and dried over MgSO₄. Evaporation of the solvent to dryness afford 41 as a white anamorphous solid (740 mg, 1.1 mmol, 99%)

### Step 2g: S-cyanomethyl 2-N-acetimido-6-O-Acetyl-3-O-[3,4,6-tri-O-acetyl-β-D-galactopyranose]-2-deoxy-1-thio-β-D-glucopyranoside 42

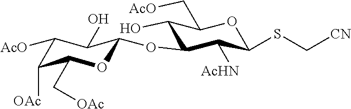

To 41 (683 mg, 1.0 mmol, 1 eq), pre-dried 30 min under high vacuum, in dry DCM (10 mL) and dry methanol (10 mL) under argon was added NH₂NH₂·AcOH (99 mg, 1.1 mmol, 1.1 eq). After 2 h more hydrazine acetate was added (10 mg, 0.1 mmol, 0.1 eq) and the reaction was stirred for another 2 h. Solvent were evaporated and the crude mixture was purified by flash chromatography on silica (biotage gradient, 0% to 10% EtOH in CHCl₃) to afford 42 as a white anamorphous solid (435 mg, 0.7 mmol, 70%)

### Step 2h: S-cyanomethyl 2-N-acetamido-6-O-acetyl-4-O-[2,3,4-tri-O-p-methoxybenzyl-6-deoxy-α-L-galactopyranosyl]-3-O-[2-(2,3,4-tri-O-p-methoxybenzyl-6-deoxy-α-L-galactopyranosyl)-3,4,6-tetra-O-acetyl-β-D-galactopyranosyl]-2-deoxy-1-thio-β-D-glucopyranoside 43

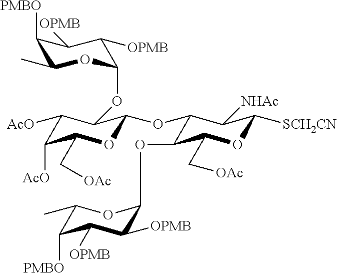

42 (300 mg, 495 μmol, 1.0 eq) and 22 (prepared in accordance with Example 1; 1125 mg, 1978 μmol, 4.0 eq) (co-evaporated 3× with toluene and dried under high vacuum for 1 h) were dissolved in dry DCM:DMF 1:1 mixture (2.0 mL) and MS 4 Å (495 mg) and stirred 1 h at rt before copper(II) bromide (442 mg, 1978 μmol, 4.0 eq) and tetra-butylammonium bromide (670 mg, 1978 μmol, 2.1 eq) were added. The mixture was stirred at rt overnigth with light exclusion. The crude mixture was filtered over celite and washed with EtOAc (200 mL). The filtrate was washed with NaHCO₃ sat. aqueous solution (5×100 mL) and brine (100 mL). The aqueous layer was re-extracted with EtOAc (200 mL). The combined organic layers where dried over MgSO₄, filtered, concentrated in vacuum and purified by flash chromatography (biotage gradient, 50% to 100% EtOAc in PE) to afford 43 as a white cream foam (617 mg, 381 μmol, 77%)

### Step 2i: S-cyanomethyl 2-N-acetamido-6-O-acetyl-4-O-[2,3,4-tri-O-acetyl-6-deoxy-β-L-galactopyranosyl]-3-O-[2-(2,3,4-tri-O-acetyl-6-deoxy-β-L-galactopyranosyl)-3,4,6-tetra-O-acetyl-β-D-galactopyranosyl]-2-deoxy-1-thio-β-D-glucopyranoside 44

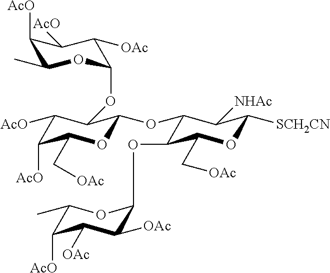

To 43 (565 mg, 349 μmol, 1.0 eq) dissolved in a mixture of MeCN:H₂O (9:1, 14 mL) was added CAN (2.3 g, 4.2 mmol, 12.5 eq). The solution was stirred at r.t. for 3 h before more CAN (190 mg, 347 μmol, 1.0 eq) was added. The reaction was stirred for another 4 h before acetic anhydride (26 mL), Pyridine (26 mL) and DMAP (4.3 mg, 35 μmol, 0.1 eq) were added. The mixture was stirred ON at r.t. before MeOH was added to quench the reaction. Evaporation of the solvant (with co-distillation with toluene 3 times) and direct purification by flash chromatography over silica (biotage, 70% to 100% EtOAc in PE) afford 44 as a white anamorphous solid (252 mg, 219 μmol, 63%)

### Step 2j: S-cyanomethyl 2-N-acetamido-4-O-[6-deoxy-α-L-galactopyranosyl]-3-O-[2-O-[6-deoxy-α-L-galactopyranosyl]-β-D-galactopyranosyl]-2-deoxy-1-thio-β-D-glucopyranoside 24

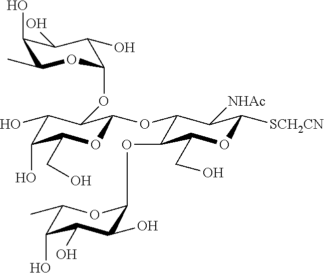

Acylated modified Le^{b} 44 (10.0 mg, 8.37 μmol) was disolved in MeOH (83.7 μL). MeONa (25% solution, 1.913 L) was added and the mixture was stirred for 10 min at room temperature. The reaction was quenched by introduction of ion exchange resin (Dowex, 100-200 mesh). After filtration and concentration (N₂ flow), the modified Le^{b} 24 (7.7 mg, 6.5 μmol, 77%) was isolated as an amorphous white powder. ¹H NMR (700 MHz, MeOD) δ 5.13 (d, J=3.3 Hz, 1H, H′″-1), 5.05 (d, J=4.0 Hz, 1H, H″-1), 4.82 (q, J=6.6 Hz, 1H, H″-5), 4.62 (d, J=7.2 Hz, 1H, H′-1), 4.59 (d, J=10.1 Hz, 1H, H-1), 4.34 (q, J=6.6 Hz, 1H, H′″-5), 4.08 (virt. t, J=9.6 Hz, 1H, H-3), 4.02 (virt. t, J=10.1 Hz, 1H, H-2), 3.96 (dd, J=12.4, 2.3 Hz, 1H, H-6a), 3.93 (dd, J=12.4, 3.6 Hz, 1H, H-6b), 3.87 (dd, J=10.2, 3.3 Hz, 1H, H″-3), 3.84 (d, J=17.2 Hz, 1H, SCHCN), 3.81 (dd, J=11.6, 7.5 Hz, 1H, H′-6a), 3.79 (virt. t, J=9.4 Hz, 1H, H-4), 3.77 (dd, J=10.2, 4.0 Hz, 1H, H″-2), 3.75 (br s, 1H, H″-4), 3.74-3.70 (m, 3H, H′″-2, H′″-3, H′″-4), 3.69-3.67 (m, 1H, H-6b), 3.68 (dd, J=10.1, 6.0 Hz, 1H, H′-2), 3.65 (dd, J=9.2, 3.0 Hz, 1H, H′-3), 3.62 (d, J=16.9 Hz, 1H, SCHCN), 3.47 (dt, J=9.6, 2.9 Hz, 1H, H-5), 3.47 (dd, J=7.1, 3.7 Hz, 1H, H′-5), 2.02 (s, 3H, NHAc), 1.28 (d, J=6.6 Hz, 3H, H″-6), 1.26 (d, J=6.6 Hz, 3H, H′″-6). ¹³C NMR (126 MHz, MeOD) δ 173.2 (NHC(═O)CH₃), 118.5 (CN), 102.5 (C′-1), 101.7 (C′-1), 99.7 (C″-1), 85.8 (C-1), 82.2 (C-5), 78.5 (C′-2), 77.8 (C-3), 76.9 (C′-3), 75.8 (C′-5), 73.8 (C″-4, C′″-4), 73.4 (C-4), 71.4 (C′″-3), 71.3 (C″-3), 70.4 (C′″-2)), 70.3 (C′-4), 70.0 (C″-2), 67.9 (C″-5), 67.3 (C′″-5), 62.9 (C′-6), 61.3 (C-6), 55.5 (C-2), 23.0 (NHC(═O)CH₃), 16.6 (C″-6), 16.6 (C′″-6), 15.1 (SCH₂CN). HRMS (m/z): calcd for C₂₈H₄₆N₂NaO₁₈S, 753.2359 ([M+Na]⁺). Found 753.2350.

### Step 2k: Mixture of S-cyanomethyl 2-N-acetamido-4-O-[6-deoxy-α-L-galactopyranosyl]-3-O-[2-O-[6-deoxy-α-L-galactopyranosyl]-β-D-galactopyranosyl]-2-deoxy-1-thio-β-D-glucopyranoside 24 and S-2-imino-2-methoxyethyl 2-N-acetamido-4-O-[6-deoxy-α-L-galactopyranosyl]-3-O-[2-O-[6-deoxy-α-L-galactopyranosyl]-β-D-galactopyranosyl]-2-deoxy-1-thio-β-D-glucopyranoside 46

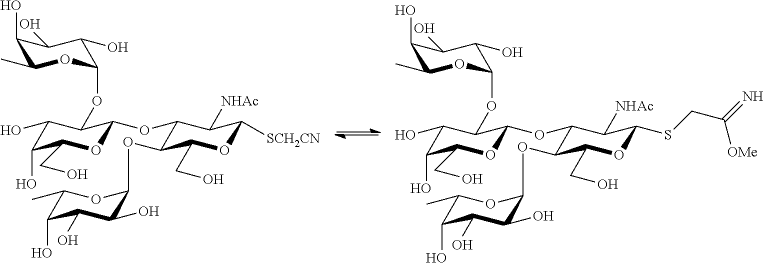

Acylated modified Le^{b} 44 (8.8 mg, 7.3 μmol) was dissolved in anhydrous MeOH (100.5 μL) under argon. NaOMe (1M, 8.2.94 μL, 0.4 equiv) was added and the mixture was stirred for 5 d at 4° C. After concentration (N₂ flow for 30 min and high vacuum for 1 h), a mixture of the activated oligosaccharide S-2-imino-2-methoxyethyl 2-N-acetamido-4-O-[6-deoxy-α-L-galactopyranosyl]-3-O-[2-O-[6-deoxy-α-L-galactopyranosyl]-β-D-galactopyranosyl]-2-deoxy-1-thio-β-D-glucopyranoside 46 and of the non-activated oligosaccharide S-cyanomethyl 2-N-acetamido-4-O-[6-deoxy-α-L-galactopyranosyl]-3-O-[2-O-[6-deoxy-α-L-galactopyranosyl]-β-D-galactopyranosyl]-2-deoxy-1-thio-β-D-glucopyranoside 24 was obtained (6.1 mg, 44 mol % and 45 mass % of 46).

### Example 3: Binding to E-Selectin

A selective blockade of E- and P-Selectin was carried out to assess the ability of Lewis A and Lewis B to bind to these two cell adhesion molecules. This was carried out using imaging agents comprising magnetic iron oxide particles conjugated to Lewis A or Lewis B.

The imaging agents used were amine-terminated MPIOs (Solulink, NanoLink™ Amino Magnetic Beads, 741 nm±88 nm diameter (current supplier TriLink Bioctehchnologies) functionalised with LewisA or LewisB using amine-reactive IME-chemistry. The magnetic beads were made up of a polystyrene core, surrounded with magnetite, and finally covered with a non polystyrene shell. The beads were functionalized with amino groups and had a hydrodynamic size of 732 nm, and a content of magnetite (Fe₃O₄) of 40%.

The magnetic particles (5.8 mgFe) were washed 3 times with the reaction buffer (NaHCO₃ 0.1 M pH 8) using a magnet support and were suspended in 0.5 mL of reaction buffer. The mixture of activated and non activated oligosaccharides, prepared as described in Examples 1 and 2 (5 to 10 equiv relative to the activated oligosaccharide) was previously solubilised in the reaction buffer (0.5 mL) and then introduced in the suspension of the magnetic particles. The mixture was shaken for 16 h at room temperature before washing the magnetic particles 3 times with MilliQ water using a magnetic support. The particles were dialyzed against MilliQ water (10 kDa MWCO), were washed 3 times with filtered (0.2 μm) MilliQ water and were suspended in filtered (0.2 μm) MilliQ water (final concentration between 4-8 mgFe/mL). LewisA loading achieved was 49%, LewisB loading achieved was 54% for the MPIOs.

To assess the binding of the LewisA and LewisB oligosaccharides in C57BL/6 mice, a tail vein injection was performed in each animal using either the E-Selectin antibody RME-1 (mouse monoclonal IgG1, κ, 3.3 mg/kg), P-Selectin antibody RMP-1 (mouse monoclonal IgG2a, κ, 3.3 mg/kg) or a mixture of the isotype control antibodies (mouse monoclonal IgG1/IgG2a, κ, 3.3 mg/kg of each, 6.6 mg in total) in a total volume of 100 μl in sterile phosphate buffered saline, pH 7.4. Immediately after the tail vein injection, IL-1β activation was carried out via a stereotactic injection of IL-1β (1 μL, 100 ng in sterile saline) in the left brain hemisphere at 1 mm anterior, 3 mm lateral from Bregma and at a 4 mm depth into the left striatum. 4 hours post IL-1β activation and antibody administration, either LewisA or LewisB magnetic particles of iron oxide (MPIO) produced as described above (4 mg/kg in sterile saline in a total volume of 100 μl) was injected intravenously in the tail vein. 1 hour post MPIO administration, animals were saline perfused and 4% paraformaldehyde (PFA) fixed.

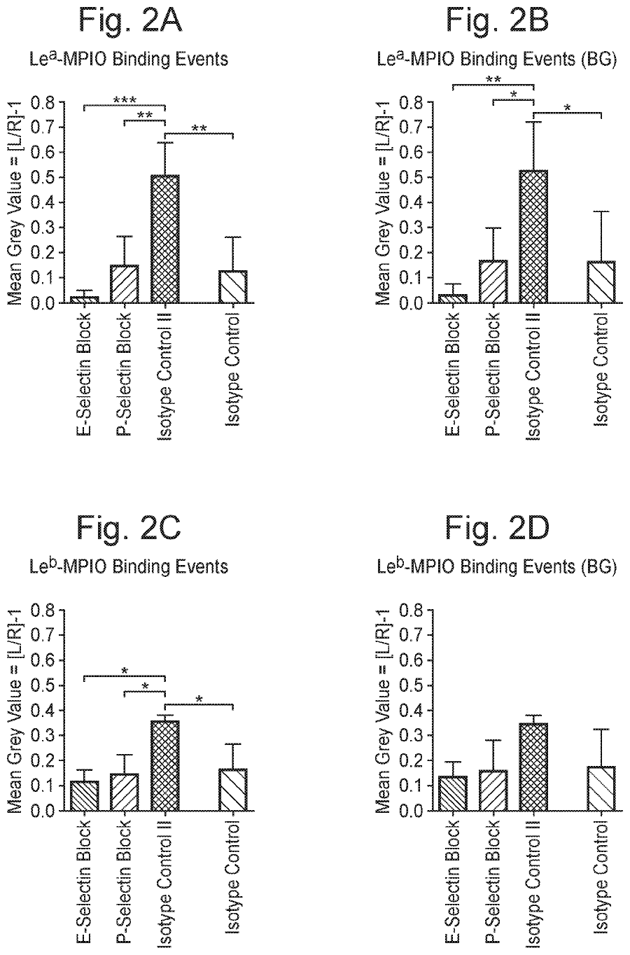

The brains were used for T2*-weighted MRI imaging on a 9.4T MRI machine. Image analysis compares the left and right hemisphere of the regions of interest. The mean grey values were compared [(Mean Grey Value Right Hemisphere/Mean Grey Value Left Hemisphere)−1]. Results are plotted in FIG. 2. The quantified binding events mean that for the lower the values, fewer Lewis-MPIOs are found in the left (activated) hemisphere. Blocking E-Selectin for LewisA indicates almost complete abolishment of LewisA-MPIO binding. For LewisB, there is residual binding present in the E-Selectin blocked animals, indicating that LewisB-MPIO binds P-Selectin as well. Although LewisB binds both E- and P-selectin, the overall binding (when LewisA and LewisB Isotype Control II group are compared) is greater for LewisA.

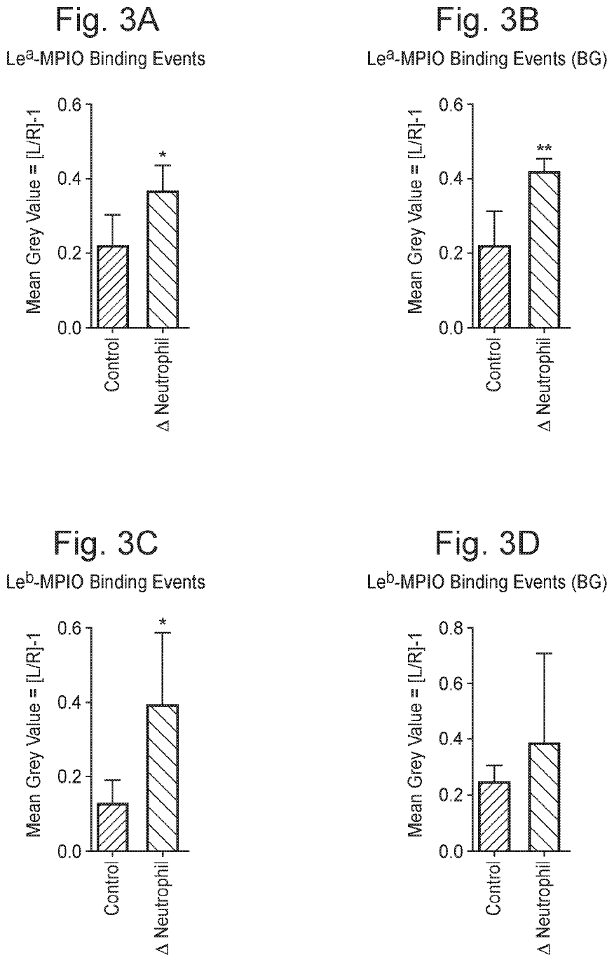

FIG. 3 provides similar results comparing binding events in control with neutrophil depleted mice.

### Example 4: Production of Sugar-Retargeted Liposomes

300 nm labelled liposomes made up of DOPC and cholesterol (CHOL), labelled with rhodamine DUPE (Texas Red® DUPE; TR-DHPE) were commercially obtained from FormuMax Scientific Inc (F60103F3-R). Liposomes, +/−DUB inhibitors, were incubated with Lewis A-lipid, +/−glutathionine for BBB-penetrating properties. Inhibitor loading was assessed using chemical analysis methods including quantitative chromatography for lipid and drug modifications. Physical properties such as size and density of the liposomes was assessed using Tunable Resistive Pulse sensing (TRPS) (qNano).

Lewis A lipids were commercially obtained from KODE Biotech Materials Limited via Sigma Aldrich (FSL-Le^{a} (tri): F9682). The molar ratio of materials used was DOPC/CHOL/TR-DHPE/Le^{a}-lipid in a ratio of 48:40:1:11 mol/mol

### Example 5: Accumulation in Brain Cells of LewisA Liposomes

A focal IL-1β-induced activation of the brain endothelium was performed in C57BL/6 mice in the manner described above in Example 3. 4 hours post IL-1β-activation, 1.25 mg of liposome (50 mg/kg) was administered intravenously in the tail vein. Liposomes administered were (a) LewisA labelled liposomes produced as described in Example 4; or (b) unlabelled (control liposomes)).

The animals receiving the LewisA labelled liposomes thus received a bolus of:
1.40 moles of DOPC: 1,2-dioleoyl-sn-glycero-3-phosphocholine
1.17 moles of CHOL: cholesterol
20 nmoles of Texas Red® DUPE
342 nmoles of Lewis^{a}-lipid

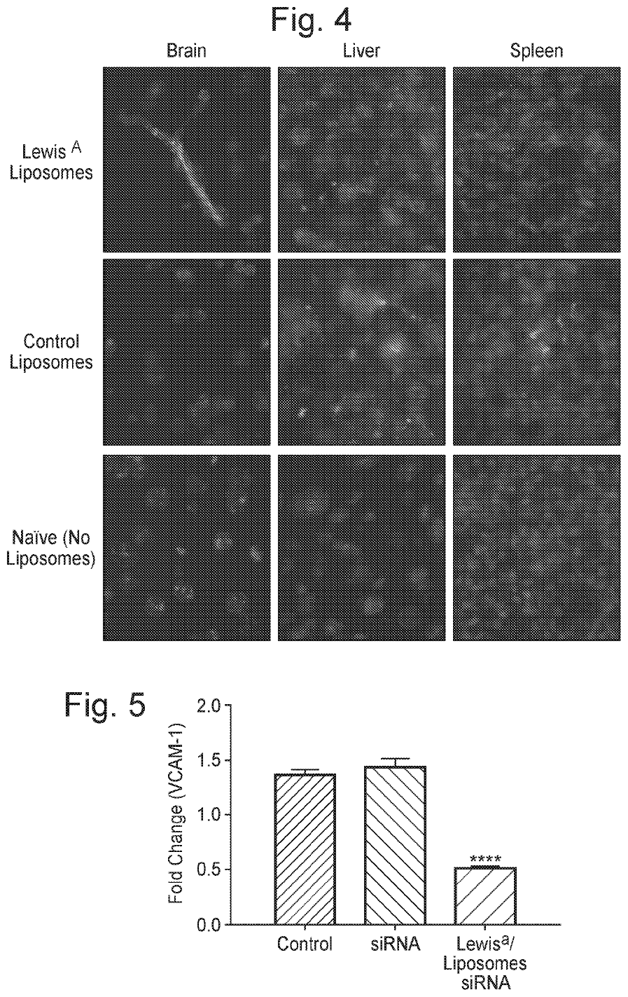

The naïve control was not administered with any liposomes. The animals were culled after 30 minutes, saline perfused and organs immediately frozen. 10 μm sections were cut and analysed by fluorescence microscopy. Results are depicted in FIG. 4. Red (light grey)=accumulation of Texas Red-liposomes. Blue (dark grey)=DAPI nuclear stain.

Accumulation of liposomes can be seen in the brains of mice administered with the LewisA labelled liposomes but not in those administered with the control liposomes. Liposomes in the LewisA mice accumulated in brain cells. Liposomes can be found in both liver and spleen of both groups. No staining is seen in any organs for the naïve mouse.

### Example 6: Delivery of VCAM-1 siRNA to Mouse Brain Endothelium

300 nm DOPC/CHOL Liposomes labelled with Texas-Red-PE were used with a lipid composition of DOPC/CHOL/Texas-Red-DHPE (54:45:1 mol/mol). Liposomes were labelled with Lewis^{a} by incubating 0.5 mg Lewis^{a}-lipid solution together with 1.5 mg of liposomes, for 18 hours at 4° C. After incubation, Lewis^{a}-labelled liposomes were transfected with VCAM-1 siRNA (SMARTpool: ON-TARGET plus VCAM1 siRNA (Dharmacon)), at a final concentration of 25 nM concentration, using an Exofect Transfection kit. Briefly, the Lewis^{a}-labelled liposomes were incubated with the Exofect transfection reagent and siRNA at 37° C. for 10 minutes. The reaction was stopped by adding the Exoquick-TC reagent and putting on ice for 30 minutes. Transfected liposomes were then centrifuged for 3 minutes at 13,000 rpm, supernatant was removed, and liposome pellet was re-suspended in sterile saline.

Activation of mouse brain endothelium was achieved by application of a 4 ng dose of murine derived IL-1β, 2 hours after application of the transfected Lewis^{a} liposomes. Liposomes were allowed to circulate for 24 hours before freezing of samples for RNA extraction. RNA extraction was performed using RNeasy Mini Kit and converted to cDNA using high capacity cDNA reverse transcription kit (Thermo-fisher). RT-qPCR was performed using a LightCycler® 480 Instrument II (Roche) to quantify VCAM-1 mRNA levels, using GAPDH (PrimerDesign) as a house-keeping gene and Primerdesign SYBR green master mix. The final reaction volume was 101. The VCAM-1 primers used were PrimePCR SYBR Green Assay (Bio-rad, qMmuCID0005772). Relative quantification was performed using ΔΔCt method and one-way ANOVA performed using GraphPad Prism.

Results are shown in FIG. 5. Compared to controls the Lewis^{a}-labelled DOPC/CHOL/Texas-Red-DHPE liposomes significantly reduced VCAM-1 mRNA expression in the brain endothelial cells.

1-20. (canceled)

21. A method for the manufacture of a targeted liposome, the method comprising binding an oligosaccharide to a lipid to provide an oligosaccharide-lipid conjugate, and combining the oligosaccharide-lipid conjugate and a non-targeted lipid in a molar ratio of from 10:90 to 80:20 to form a targeted liposome; wherein the oligosaccharide is Lewis A or Lewis B or a mimetic thereof, or a pharmaceutically acceptable salt or PEGylated form of the oligosaccharide: 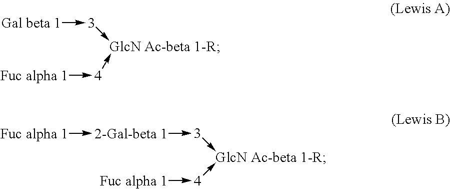
wherein R represents the point of attachment to the lipid.

22. A method according to claim 21, wherein the mimetic of Lewis A or Lewis B is Lewis A or Lewis B having one or more of the following modifications (i) to (iv):
(i) one or more OH and/or NAc groups are independently replaced with hydrogen, halogen, C₁₋₆ alkoxy, —NR′R″, —NR′COR″, —N(COR′)(COR″), —SR′, —COR′, —COOR′, —OC(O)R′, —OC(O)OR′, —OC(O)NR′R″, —OC(O)SR′, —OP(O)(OR′)(OR″), or C₁₋₁₂ alkyl, C₂₋₁₂ alkenyl or C₂₋₁₂ alkynyl which is optionally substituted with one or more substituents selected from halogen, NH₂, N₃, CN, COOH, COO(C₁₋₄ alkyl), OH and C₁₋₄ alkoxy, or in the case of NAc may be replaced with OH;
(ii) one or more hydrogen atoms are independently replaced with OH, halogen or a group selected from OR′, —NR′R″, —SR′, —COR′, —COOR′, —OC(O)R′, —OC(O)OR′, —OC(O)NR′R″, —OC(O)SR′, —OP(O)(OR′)(OR″), or C₁₋₁₂ alkyl, C₂₋₁₂ alkenyl or C₂₋₁₂ alkynyl which is optionally substituted with one or more substituents selected from halogen, NH₂, N₃, CN, COOH, COO(C₁₋₄ alkyl), OH and C₁₋₄ alkoxy;
(iii) one or more —O— moieties are independently replaced with —CR′R″—, —S—, —NR′— or —NR′CO—;
(iv) in the Glc saccharide unit, the unit —C₁(R)—O— is replaced with —C₁(R)═N— or —C₁(R)═CR′—;
wherein each R′ and R″ is identical or different and is selected from hydrogen and C₁₋₁₂ alkyl groups which are optionally substituted with one or more substituents selected from halogen, NH₂, N₃, CN, COOH, COO(C₁₋₄ alkyl), OH and C₁₋₄ alkoxy.

23. A method according to claim 21, wherein the mimetic of Lewis A or Lewis B contains one, two, three or four modifications compared with the Lewis A or Lewis B itself.

24. A method according to claim 21, wherein the mimetic of Lewis A or Lewis B is Lewis A or Lewis B having one, two or three of the following modifications:
(i) one, two or three OH and/or NAc groups are independently replaced with hydrogen, halogen, methoxy, ethoxy, —NH₂, (C₁₋₂alkyl)amine, di(C₁₋₂alkyl)amine, (C₁₋₂acetyl)amine, di(C₁₋₂acetyl)amine, mercapto, methylthio, ethylthio or —OP(O)(OH)₂;
(ii) one, two or three hydrogen atoms are independently replaced with fluorine;
(iia) the axial and equatorial positions on one, two or three carbon atoms are reversed;
(iib) one, two or three carbon atoms are disubstituted such that both the OH and hydrogen are replaced with a group which is independently selected from halogen, methoxy, ethoxy, —NH₂, (C₁₋₂alkyl)amine, di(C₁₋₂alkyl)amine, (C₁₋₂acetyl)amine, di(C₁₋₂acetyl)amine, mercapto, methylthio, ethylthio or —OP(O)(OH)₂;
(iii) one or more —O— moieties are independently replaced with —SH—, —NH— or —N(Me)-;
(iv) in the Glc saccharide unit, the unit —C₁(R)—O— is replaced with —C₁(R)═N—.

25. A method for the manufacture of a targeted liposome, the method comprising binding an oligosaccharide to an amine group on a lipid to provide an oligosaccharide-lipid conjugate, and combining the oligosaccharide-lipid conjugate and a non-targeted lipid in a molar ratio of from 10:90 to 80:20 to form a targeted liposome;
wherein the oligosaccharide is of formula (I), or a pharmaceutically acceptable salt or PEGylated form of the oligosaccharide: 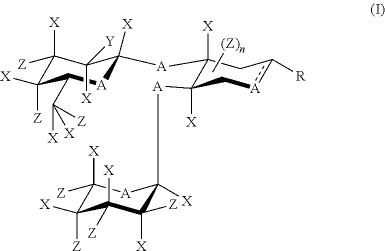
wherein each Z is the same or different and is selected from OH, hydrogen, halogen,
C₁₋₆ alkoxy, —NR′R″, —NR′COR″, —N(COR′)(COR″), —SR′, —COR′, —COOR′, —OC(O)R′, —OC(O)OR′, —OC(O)NR′R″, —OC(O)SR′, —OP(O)(OR′)(OR″), or C₁₋₁₂ alkyl, C₂₋₁₂ alkenyl or C₂₋₁₂ alkynyl, which is optionally substituted with one or more substituents selected from halogen, NH₂, N₃, CN, COOH, COO(C₁₋₄ alkyl), OH and C₁₋₄ alkoxy;
each X is the same or different and is selected from OH, hydrogen, halogen,
C₁₋₆ alkoxy, —NR′R″, —NR′COR″, —N(COR′)(COR″), —SR′, —COR′, —COOR′, —OC(O)R′, —OC(O)OR′, —OC(O)NR′R″, —OC(O)SR′, —OP(O)(OR′)(OR″), or C₁₋₁₂ alkyl, C₂₋₁₂ alkenyl or C₂₋₁₂ alkynyl, which is optionally substituted with one or more substituents selected from halogen, NH₂, N₃, CN, COOH, COO(C₁₋₄ alkyl), OH and C₁₋₄ alkoxy;
each A is the same or different and is selected from CR′R″, O, S and NR′;
n is 0, 1, 2, 3 or 4;
Y is either a group Z as defined above or a saccharide unit of formula (II): 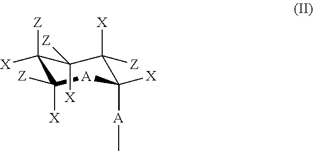
wherein X, Z and A are as defined above;
R represents the point of attachment to the lipid; and
R′ and R″ are identical or different and are selected from hydrogen and C₁₋₂ alkyl groups which are optionally substituted with one or more substituents selected from halogen, NH₂, N₃, CN, COOH, COO(C₁₋₄ alkyl), OH and C₁₋₄ alkoxy.

26. A method according to claim 25, wherein either all groups Z are OH, or from one to six groups Z are other than OH and the remainder represent OH; either all groups X are hydrogen or one, two or three groups X are other than hydrogen and the remainder represent hydrogen; either each A is —O— or one, two or three A groups are other than —O— and the remainder represent —O—; and n is 0, 1, 2 or 3.

27. A method according to claim 25, wherein each Z is the same or different and is selected from OH, CH₂OH, hydrogen, halogen, methyl, methoxy, ethoxy, —NH₂, (C₁₋₂alkyl)amine, di(C₁₋₂alkyl)amine, (C₁₋₂acetyl)amine, di(C₁₋₂acetyl)amine, mercapto, methylthio, ethylthio and —OP(O)(OH)₂.

28. A method according to claim 25, wherein each X is the same or different and is hydrogen or halogen.

29. A method according to claim 25, wherein where a carbon atom carries a group Z which is other than OH, then X on the same carbon atom represents OH, hydrogen, halogen, methoxy, ethoxy, —NH₂, (C₁₋₂alkyl)amine, di(C₁₋₂alkyl)amine, (C₁₋₂acetyl)amine, di(C₁₋₂acetyl)amine, mercapto, methylthio, ethylthio or —OP(O)(OH)₂.

30. A method according to claim 25, wherein each A is the same or different and is —O—, —SH—, —NH— or —N(Me)-.

31. A method according to claim 25, wherein the oligosaccharide is of formula (Ia) or (Ib) or a pharmaceutically acceptable salt or PEGylated form thereof: 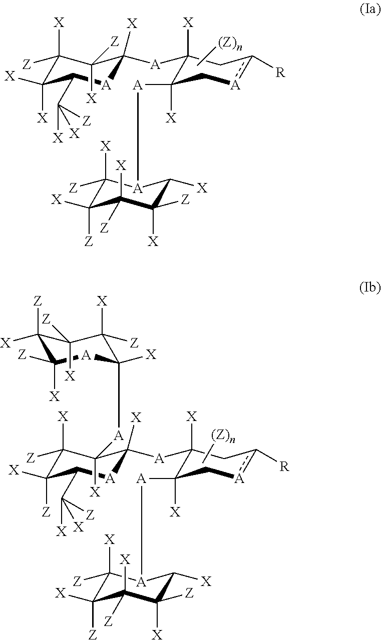
wherein:
each Z is the same or different and is selected from OH, CH₂, OH, hydrogen, methyl, halogen, methoxy, ethoxy, —NH₂, (C₁₋₂alkyl)amine, di(C₁₋₂alkyl)amine, (C₁₋₂acetyl)amine, di(C₁₋₂acetyl)amine, mercapto, methylthio, ethylthio and —OP(O)(OH)₂, wherein either all groups Z are OH, or from one to six groups are other than OH and the remainder are OH;
each X is the same or different and is selected from hydrogen or halogen, or in the case where a group Z carried on the same carbon atom as X is other than OH, then X represents OH, hydrogen, halogen, methoxy, ethoxy, —NH₂, (C₁₋₂alkyl)amine, di(C₁₋₂alkyl)amine, (C₁₋₂acetyl)amine, di(C₁₋₂acetyl)amine, mercapto, methylthio, ethylthio or —OP(O)(OH)₂, wherein either all groups X are hydrogen, or one or two groups are other than hydrogen and the remainder are hydrogen;
each A is the same or different and is selected from —O—, —SH—, —NH— and —N(Me)-; wherein either all groups A are —O— or one A group is other than —O— and the remainder represent —O—;
n is 2;
the bond C₁(R)-A is saturated; and
R represents the point of attachment to the lipid.

32. A method according to claim 21, wherein the oligosaccharide is Lewis A or Lewis B or a pharmaceutically acceptable salt thereof.

33. A method according to claim 21, wherein the molar ratio of oligosaccharide-lipid conjugate to non-targeted lipid is from 20:80 to 80:20.

34. A method according to claim 21, wherein the molar ratio of oligosaccharide-lipid conjugate to non-targeted lipid is from 60:40 to 80:20.

35. A method according to claim 21, wherein the oligosaccharide is bound to an amine group on the lipid.

36. A method according to claim 21, wherein the liposome has a particle size of from 100 to 800 nm.

37. A method according to claim 21 wherein the liposome further contains a therapeutic agent.

38. A method according to claim 37, wherein the therapeutic agent is a deubiquitinating enzyme inhibitor.
